(19) United States
(12) Patent Application Publication
(10) Pub. No.: US 2026/0309990 A1
(43) Pub. Date: Oct. 8, 2026

(54) WEARABLE OPTICAL SEE-THROUGH (OST) NEAR-EYE DISPLAY (NED) SYSTEM INTEGRATING OPHTHALMIC CORRECTION WITH IMPROVED PANTOSCOPIC ANGLE

(71) Applicant: EVERYSIGHT LTD., Haifa (IL)

(72) Inventors: Hanan SHAMIR, Haifa (IL); Giora YAAHAV, Haifa (IL); Asaf ASHKENAZI, Haifa (IL)

(73) Assignee: EVERYSIGHT LTD., Haifa (IL)

(21) Appl. No.: 19/480,721

(22) PCT Filed: May 3, 2024
(86) PCT No.: PCT/IL2024/050425

§ 371 Date: Oct. 31, 2025

Related U.S. Application Data
(60) Provisional application No. 63/463,611, filed on May 3, 2023.
(60) Provisional application No. 63/641,444, filed on May 2, 2024.

(51) Int. Cl. G02B 27/01; G02B 1/11; G02B 17/08; G02C 7/02

(52) U.S. Cl. CPC G02B 27/0172; G02B 1/11; G02B 17/08; G02C 7/022; G02B 2027/011; G02B 2027/013; G02B 2027/014; G02B 2027/0174; G02B 2027/0178; G02B 2027/0196

(57) ABSTRACT

An optical see-through (OST) near-eye display (NED) off-axis system, providing ophthalmic correction to an eye of a user, for improving a pantoscopic angle in a system based on a glabellar region positioned electro-optical sub-system, the system includes a partially transmissive partially reflective lens, an intermediate reflective surface, and an electro-optical subsystem, the partially transmissive partially reflective lens includes a material having a refractive index, an inner surface characterized by an inner surface radius of curvature exhibiting a first optical power, and an outer surface characterized by an outer surface radius of curvature exhibiting a second optical power, said partially transmissive partially reflective lens is configured to be facing said eye, and to at least partially transmit incoming light of an outward scene to said eye, said intermediate reflective surface is characterized by an intermediate reflective radius of curvature exhibiting a third optical power, said intermediate reflective surface is embedded within said partially transmissive partially reflective lens, between said inner surface and said outer surface, said electro-optical sub-system is configured to be optically coupled with said partially transmissive partially reflective lens, said electro-optical sub-system including a light display configured to project a light beam image onto said intermediate reflective surface enabling reflection of said light beam image toward said eye, said electro-optical sub-system is configured to be located at a glabellar region of said user, wherein said first optical power and said second optical power are configured in conjunction with said material refractive index to provide ophthalmic correction with respect to transmitted said incoming light from said outward scene for viewing by said eye, wherein said third optical power is configured to provide ophthalmic correction with respect to reflected said light image for viewing by said eye, wherein said intermediate reflective surface is oriented such to have a tilt angle with respect to said at least one of said inner surface and said outer surface enabling a reduction of said pantoscopic angle.

### CROSS-REFERENCE TO RELATED APPLICATIONS

This application is a National Stage Application of PCT/IL2024/050425, filed May 3, 2024, which claims benefit of priority to U.S. Provisional Application No. 63/641,444 filed on May 2, 2024, and to U.S. Provisional Application No.: 63/463,611, filed May 3, 2023, which applications are incorporated herein by reference. To the extent appropriate, a claim of priority is made to each of the above disclosed applications.

### FIELD OF THE INVENTION

The disclosed technique relates to optical systems in general, and to wearable optical display systems, in particular.

### BACKGROUND OF THE INVENTION

Optical head-mounted display systems, such as optical see-through (OST) display systems are user-wearable devices that allow a user to see through a see-though part of a display while concurrently enabling the user to see images displayed on the display. This type of display system allows for augmented reality (AR), in which the user sees a real-world outward scene that is combined (e.g., on a combiner) with a generated display of objects (e.g., via computer-generated data). Various types of OST display systems are known in the art. One type of OST display system is a system where an electro-optical module that displays and projects the generated objects (e.g. images) is entirely located at a glabellar region of the user, when the OST display system is worn by the user. The glabella or glabellar region in humans is defined as an anatomical area between the eyebrows and above the nose.

An example of one such OST display system based on glabellar-region positioned electro-optical module is disclosed in U.S. Patent Application Publication No.: US 2015/0168730 A1 to Ashkenazi et al. and titled “Wearable Optical Display System for Unobstructed Viewing” is directed at a user wearable optical display system that provides information in the form of projected light to a user who wears the system without obstructing the user's field of regard (FOR). The user wearable optical display system includes a user attachment section, a partially transmissive partially reflective lens, and an electro-optical unit. The user attachment section is for detachably mounting the user wearable optical display system to a head of a user. The partially transmissive partially reflective lens, which is coupled with the user attachment section, is configured to be facing at least one eye of the user. The electro-optical unit is coupled with at least one of the user attachment section and the partially transmissive partially reflective lens. The electro-optical unit includes a processor, and a light projection unit. The processor is coupled with the light projection unit. The light projection unit is configured to transmit light beams onto the partially transmissive partially reflective lens. The electro-optical unit is configured to be positioned with respect to the user attachment section such that when the user wearable optical display system is mounted on the user, the electro-optical unit is located at the glabellar region of the user. The positioning of the electro-optical unit at the glabellar region provides a substantially unobstructed FOR for the user.

### SUMMARY OF THE INVENTION

It is an object of the disclosed technique to provide a novel optical see-through (OST) near-eye display (NED) off-axis system that provides ophthalmic correction to an eye of a user, for improving a pantoscopic angle in a system based on a glabellar region positioned electro-optical sub-system. The system includes a partially transmissive partially reflective lens, an intermediate reflective surface, and an electro-optical sub-system. The partially transmissive partially reflective lens includes a material having a refractive index, an inner surface characterized by an inner surface radius of curvature exhibiting a first optical power, and an outer surface characterized by an outer surface radius of curvature exhibiting a second optical power. The partially transmissive partially reflective lens is configured to be facing the eye, and to at least partially transmit incoming light of an outward scene to the eye. The intermediate reflective surface is characterized by an intermediate reflective radius of curvature exhibiting a third optical power. The intermediate reflective surface is embedded within the partially transmissive partially reflective lens, between the inner surface and the outer surface. The electro-optical sub-system is configured to be optically coupled with the partially transmissive partially reflective lens. The electro-optical sub-system includes a light display configured to project a light beam image onto the intermediate reflective surface enabling reflection of the light beam image toward the eye. The electro-optical sub-system is configured to be located at a glabellar region of the user. The first optical power and the second optical power are configured, in conjunction with the material refractive index, to provide ophthalmic correction with respect to the transmitted incoming light from the outward scene for viewing by the eye. The third optical power is configured to provide ophthalmic correction with respect to the reflected light beam image for viewing by the eye, whereby the intermediate reflective surface is oriented such to have a tilt angle with respect to at least one of the inner surface and the outer surface enabling a reduction of the pantoscopic angle.

It is a further object of the disclosed technique to provide a novel optical see-through (OST) near-eye display (NED) off-axis system providing ophthalmic correction to an eye of a user for improving a pantoscopic angle in a system based on a glabellar region positioned electro-optical sub-system. The system includes a partially transmissive partially reflective lens, an intermediate reflective surface, and an electro-optical sub-system. The partially transmissive partially reflective lens includes a material having a refractive index, an inner surface, and an outer surface. The partially transmissive partially reflective lens is configured to be facing the eye, and to at least partially transmit incoming light of an outward scene to the eye. The intermediate reflective surface is embedded within the partially transmissive partially reflective lens and is positioned between the inner surface and the outer surface. The electro-optical sub-system is configured to be optically coupled with the partially transmissive partially reflective lens so as to project a light beam image onto the intermediate reflective surface. The electro-optical sub-system is configured to be located at a glabellar region of the user. The electro-optical sub-system includes a light display, a first lens, a second lens, a third lens, and a fourth reflecting lens. The light beam image propagates along an optical path from the light display toward the third lens, then toward the fourth reflecting lens, then again toward the third lens, then toward the second lens, then toward the first lens, then toward the partially transmissive partially reflective lens.

### BRIEF DESCRIPTION OF THE DRAWINGS

The disclosed technique will be understood and appreciated more fully from the following detailed description taken in conjunction with the drawings in which:

FIG. 1 is a schematic illustration of an optical see-through (OST) near-eye display (NED) system, constructed and operative in accordance an embodiment of the disclosed technique;

FIG. 2 is a schematic illustration of the OST NED system of FIG. 1 in an exploded view;

FIG. 3A is a schematic block diagram of a basic configuration of the electro-optical unit of OST NED system, constructed and operative in accordance with the embodiment of the disclosed technique;

FIG. 3B is a schematic block diagram of an accessorized configuration of the electro-optical unit of OST NED system, constructed and operative in accordance with the embodiment of the disclosed technique;

FIG. 4 is a schematic illustration showing OST NED system of FIGS. 1 and 2 in a mounted configuration on a user;

FIG. 5 is a schematic diagram of a light path through an optical module of electro-optical unit of OST, constructed and operative in accordance with the embodiment of the disclosed technique;

FIG. 6 is a schematic illustration showing a pantoscopic angle, a wrap angle, a bend angle, and a roll angle according to the principles of the disclosed technique;

FIG. 7A is an illustration showing a schematic diagram of a light path through a glabellar-region positioned electro-optical subsystem in an optical see-through (OST) near-eye display (NED) off-axis system for improving a pantoscopic angle, constructed and operative in accordance with an embodiment of the disclosed technique;

FIG. 7B is an illustration showing a schematic diagram of a light path through a glabellar-region positioned electro-optical subsystem in an optical see-through (OST) near-eye display (NED) off-axis system for improving a pantoscopic angle, constructed and operative in accordance with another embodiment of the disclosed technique;

FIGS. 8A to 8C are schematic illustrations of the partially transmissive partially reflective lens, in several viewpoints, constructed and operative in accordance with the embodiment of the disclosed technique, particularly:

FIG. 8A shows a front view,

FIG. 8B shows a side view, and

FIG. 8C shows a top view;

FIGS. 9A to 9C are schematic illustrations of the partially transmissive partially reflective lens in FIGS. 8A-8C, showing an exploded view, constructed and operative in accordance with the embodiment of the disclosed technique, particularly:

FIG. 9A is a perspective exploded view,

FIG. 9B a side exploded view, and

FIG. 9C is top exploded view;

FIG. 10 is an illustration showing a schematic diagram of a light path through a glabella-region positioned optical module of the electro-optical unit of OST NED off-axis system for improving a pantoscopic angle, constructed and operative in accordance with a further embodiment of the disclosed technique;

FIGS. 11A to 11C are schematic illustrations of the partially transmissive partially reflective lens, in several viewpoints, constructed and operative in accordance with the embodiment of the disclosed technique, particularly:

FIG. 11A shows a front view,

FIG. 11B shows a side view,

FIG. 11C shows a top view;

FIGS. 12A to 12C are schematic illustrations detailing the structure in exploded views of the partially transmissive partially reflective lens of FIGS. 11A-11C, constructed and operative in accordance with the embodiment of the disclosed technique, particularly:

FIG. 12A shows a perspective exploded view,

FIG. 12B shows a side exploded view,

FIG. 12C shows a top exploded view;

FIG. 13 is an illustration showing a schematic diagram of a light path through a glabella-region positioned optical module of the electro-optical unit in OST NED off-axis system, providing ophthalmic correction to the user, utilizing an intermediate reflective surface embedded in the partially transmissive partially reflective lens, constructed and operative in accordance with another embodiment of the disclosed technique; and

FIG. 14 is an illustration showing a schematic diagram of a light path through a glabella-region positioned optical module of an electro-optical unit of OST NED off-axis system, constructed and operative in accordance with a further embodiment of the disclosed technique.

### DETAILED DESCRIPTION

The disclosed technique overcomes the disadvantages of the prior art by providing an optical see-through (OST) near-eye display (NED) off-axis system having ophthalmic correction to at least one eye or both eyes of a user, for improving a pantoscopic angle in a system based on a glabellar region positioned electro-optical sub-system (interchangeably denoted herein as “electro-optical unit”, “electro-optical module”, and “electro-optical subsystem”). In prior art OST NED systems based on glabellar region positioned electro-optical units, the pantoscopic angle is typically greater than the pantoscopic angle of typical wearable optical devices (e.g., eyewear such as framed glasses), since the electro-optical unit's physical volume and its placement at the glabellar region affects and limits a reduction of the pantoscopic angle afforded to the user in such systems. The approach of the disclosed technique solves this constraint by enabling to reduce (and thus “improve”) the pantoscopic angle in such systems. The pantoscopic angle is generally defined as an angle between a general vertical line and a vertical axis of the eye-wear lens connected to the eye-wear frame (the angle of the eye-wear lens relative to the temple of the eye-wear). The pantoscopic angle can be regarded as an important parameter in eyewear in general, and particularly, in wearable OST NED off-axis systems that provide ophthalmic correction to the user. A pantoscopic angle of eyewear can affect the pantoscopic tilt (i.e., the angle between the vertical plane and the lens plane in relation to the user's visual axis when worn), thereby influencing derivative aberrations including astigmatism and how the partially transmissive partially reflective lens is positioned in front of the user's visual axis (position of primary gaze). An improved or optimized pantoscopic angle in such systems (e.g., in glabellar region positioned electro-optical subsystems) can contribute to enhanced centration and image quality as well as a reduction of aberrations. The system of the disclosed technique includes a partially transmissive partially reflective lens, an intermediate reflective surface, and an electro-optical sub-system. The partially transmissive partially reflective lens includes an optical material having an index of refraction, an inner surface characterized by an inner surface radius of curvature exhibiting a first optical power, and an outer surface characterized by an outer surface radius of curvature exhibiting a second optical power. The partially transmissive partially reflective lens is configured to be facing at least one eye of the user, and is further configured to at least partially transmit incoming light of an outward scene to the eye. The intermediate reflective surface is characterized by an intermediate reflective surface radius of curvature (interchangeably denoted herein “reflective radius of curvature”, “reflective surface radius of curvature”, and “intermediate radius of curvature”) exhibiting a third optical power. The intermediate reflective surface is embedded within the partially transmissive partially reflective lens, intermediate between the inner surface and the outer surface. (The term “intermediate” with reference to the intermediate reflective surface signifies that it is positioned in-between the inner surface and the outer surface, but not necessarily at mid-distance therebetween.) The electro-optical sub-system is configured to be optically coupled with the partially transmissive partially reflective lens. The electro-optical sub-system includes a light display configured to project a light beam image onto the intermediate reflective surface enabling reflection of the light beam image toward the eye. The electro-optical sub-system is configured to be located at a glabellar region of the user. The first optical power and the second optical power are configured, in conjunction with the index of refraction of the partially transmissive partially reflective lens, to provide ophthalmic correction with respect to the transmitted incoming light from the outward scene for viewing by the eye. The third optical power is configured to provide ophthalmic correction with respect to reflected light beam image for viewing by the eye. The intermediate reflective surface is oriented such to have a tilt angle with respect to at least one of the inner surface and the outer surface enabling a reduction of the pantoscopic angle. The pantoscopic angle is defined as an angle between a general vertical line and a vertical axis of the eye-wear frame (the angle of the eye-wear frame with relative to the temple of the user).

In accordance with another aspect of the disclosed technique, there is thus provided an optical see-through (OST) near-eye display (NED) off-axis system, providing ophthalmic correction to an eye of a user, for improving a pantoscopic angle in a system based on a glabellar region positioned electro-optical sub-system. The system includes a partially transmissive partially reflective lens, an intermediate reflective surface, and an electro-optical subsystem (herein denoted interchangeably “electro-optical unit”). The electro-optical sub-system includes a light display, a first lens, a second lens, a third lens, and a fourth reflecting lens. The partially reflective partially reflective lens includes an inner surface and an outer surface. The inner surface is configured to be facing the eye, and to at least partially transmit incoming light of an outward scene to the eye. The intermediate reflective surface is embedded within the partially transmissive partially reflective lens, between the inner surface and the outer surface. The electro-optical sub-system is configured to be optically coupled with the partially transmissive partially reflective lens so as to project a light beam image onto the intermediate reflective surface. The electro-optical sub-system is configured to be located at a glabellar region of the user. The light beam image propagates along an optical path from the light display toward the third lens, then toward the fourth reflecting lens, then again toward the third lens, then toward the second lens, then toward the first lens, then toward the partially transmissive partially reflective lens.

The disclosed technique provides a free-space optical see-through (OST) near-eye display (NED) off-axis system that includes a set of optical lenses that are mechanically integrated and characterized by a tilt angle and a wrap angle that meet the optical standards for the pantoscopic and wrap angles of typical eyewear, although the electro-optical unit is configured to be located at the glabellar region of the user when worn. In applicant's prior art system described in the Background Section, the electro-optical unit is configured to be located at the glabellar region of the user when worn; however, such a configuration increases the pantoscopic angle. Such a system is denoted herein as “a system based on a glabellar region positioned electro-optical sub-system”. The disclosed technique improves on applicant's prior art system by reducing the pantoscopic angle, as well as simultaneously providing ophthalmic correction to at least one or both eyes of a user (in accordance with the user's specific ophthalmic prescription). Particularly, the intermediate reflective surface is oriented such to have a tilt angle with respect to either the inner or outer surfaces (or both) thus enabling a reduction of the pantoscopic angle.

The intermediate reflective surface is entirely incorporated into (embedded and enclosed within) the partially transmissive partially reflective lens material that is characterized by having at least one index of refraction. According to one implementation of the disclosed technique, the partially transmissive partially reflective lens material is characterized by having one index of refraction. According to an alternative implementation of the disclosed technique, the partially transmissive partially reflective lens has a plurality of different indices of refraction at its different locations (not shown). The partially transmissive partially reflective lens structure is generally characterized by its geometry and type (e.g., surfaces' radiuses of curvatures, thicknesses, etc.), its composition (material make up)), all of which determine its optical properties (e.g., optical power, index of refraction, Abbe number, etc.). According to one optical configuration, the intermediate reflective layer is considered an integral part of the partially transmissive partially reflective lens. According, to another optical configuration, the intermediate reflective layer is considered as a non-integral, add-on optical element that is combined with the partially transmissive partially reflective lens after manufacturing of the latter. There are three main radii of curvatures of interest. The partially transmissive partially reflective lens includes an inner surface characterized by an inner surface radius of curvature, an outer surface characterized by an outer surface radius of curvature and an intermediate reflective surface characterized by an intermediate reflective surface radius of curvature. Each of the surfaces of the partially transmissive partially reflective lens is tiled in specific tilt and wrap angles with respect to the optical axis, as well as with respect to each other. The tilt and wrap angles are referenced with respect to the light projection optical axis.

The electro-optical unit is optically coupled with the partially transmissive partially reflective lens, exhibiting a first tilt angle and a first wrap angle with respect to an optical axis, in accordance with the tilt and wrap angles of the intermediate reflective surface, so that the light display (light projector) that projects the light beam is reflected toward the eye. Furthermore, the electro-optical unit is optically coupled with the partially transmissive partially reflective lens, exhibiting a second tilt angle and a second wrap angle in accordance with the tilt and wrap angles of the outer and inner surfaces, so that the mechanical pantoscopic angle and wrap angle conforms with optical standards of typical eyewear. According to an embodiment of the disclosed technique, the light display that projects the light beam passes through the inner surface of the partially transmissive partially reflective lens, propagates by refraction through the lens material having an index of refraction, reflects back from the intermediate reflective surface, propagates by refraction again through the lens material, passes through the inner surface again and propagates through free-space (e.g., air) toward the eye of the user.

According to one implementation of the disclosed technique, the intermediate reflective surface is coated (e.g., covered) with a partially transmissive partially reflective mirror coating, in which the reflection angle equals to the incidence angle according to Snell's law of reflection. It may be appreciated by a person skilled in the art that once a mutual position between the eye and the light projection unit is fixed, the tilt angle and wrap angle of the reflective surface is injectively defined to allow the light beam from the light projector to be directed toward the eye.

According to another implementation of the disclosed technique, the intermediate reflective surface is constructed, structured, and operative as a diffractive reflecting grating, in which there are multiple reflection angles (orders of diffraction) in accordance with a grating equation (dependent on groove spacing). Utilization of a diffractive grating enables control of the tilt and wrap angles of the reflective surface while keeping the light beam directed at the eye, perhaps with some compromises in optical efficiency in accordance with diffraction grating efficiency.

According to a further implementation of the disclosed technique, the intermediate reflective surface is constructed, structured, and operative as a meta-surface, in which the reflection angle is in accordance with its meta-surface grating equation. It may be appreciated by a person skilled in the art that the meta-surface grating technique demonstrates an improved optical efficiency compared to a regular diffractive grating technique especially when it concerns an achromatic light beam.

According to another implementation of the disclosed technique, the intermediate reflective surface is constructed, structured, and operative specifically as a holographic grating having a holographic surface or holographic volume that may exhibit a high optical efficiency that is comparable to or better than traditional mechanically ruled diffraction gratings. The use of holographic gratings enables to reduce stray light, as well as to reduce or eliminate ghosting effects that may typically be exhibited by traditional ruled diffraction gratings.

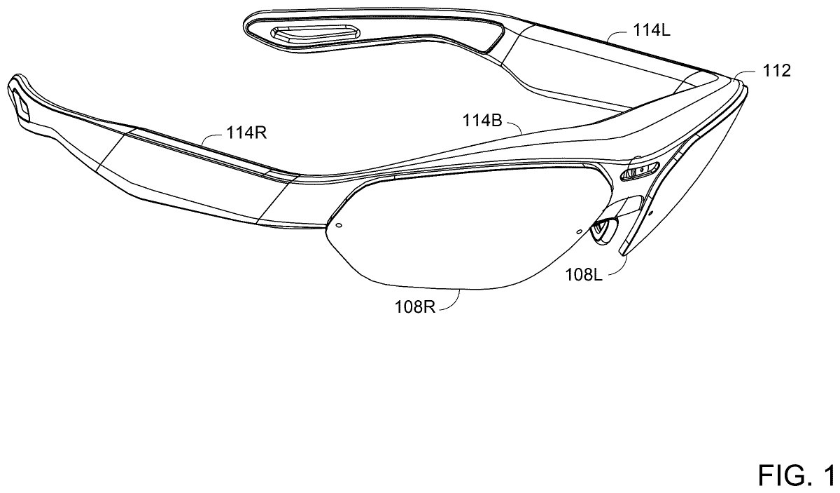

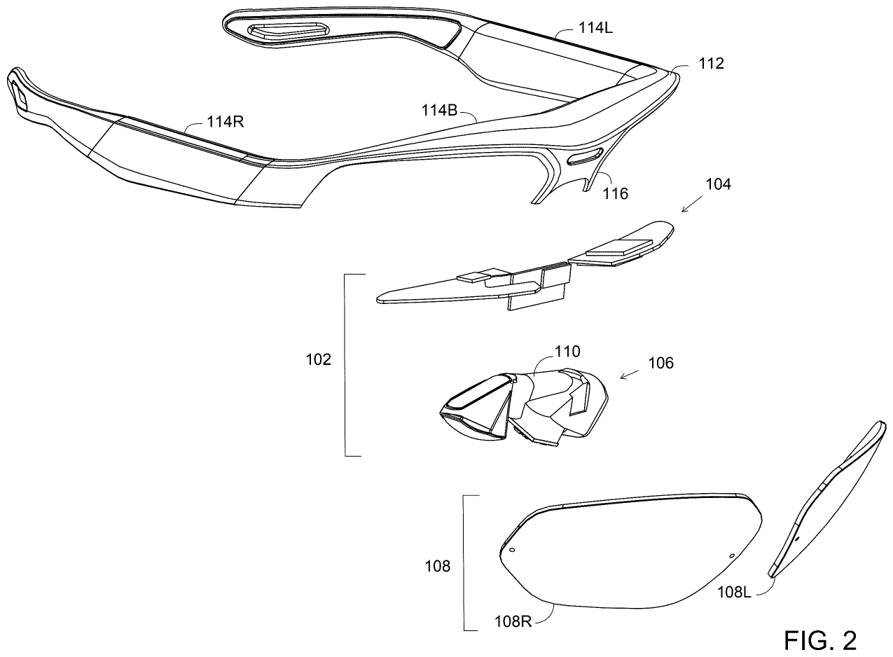

Reference is now made to FIGS. 1 and 2. FIG. 1 is a schematic illustration of an optical see-through (OST) near-eye display (NED) system, generally referenced 100, constructed and operative in accordance an embodiment of the disclosed technique. FIG. 2 is a schematic illustration of the OST NED system of FIG. 1 in an exploded view. OST NED system 100 includes an electro-optical unit 102 (interchangeably denoted herein “electro-optical sub-system”, and “electro-optical subsystem”) and at least one partially transmissive partially reflective lens 108. Electro-optical unit 102 includes an electronics module 104 (FIG. 2), and an optical module 106. Optical module 106 is configured to be at least partially housed in an optical housing 110 (FIG. 2). Electro-optical unit 102 is configured to be mechanically and optically coupled such to have fixed position and orientation with respect to at least one partially transmissive partially reflective lens 108 (i.e., and vice-versa).

FIGS. 1 and 2 show a user attachment section 112 that enables the detachable coupling of OST NED system 100 to a user (i.e., to be worn by a user). User attachment section 112 is typically embodied in the form of a frame (e.g., resembling eye glasses) and includes two temples 114R and 114L (interchangeably “stem portions”), a bridge 114B (“bridge portion”) that couples between stem portions 114L and 114R, and a nosepiece 116. The terms “user attachment section”, “eye-wear frame”, and “frame” are herein interchangeable and refer to a device, object or group of objects configured and operative to couple with a wearer of OST NED system 100. FIGS. 1 and 2 illustrate two partially transmissive partially reflective lenses, namely, a partially transmissive partially reflective lens 108R (denoted interchangeably herein simply “lens”, “combiner”, “optical combiner”), and partially transmissive partially reflective lens 108L, each configured to be facing a different eye of a user wearing OST NED system 100.

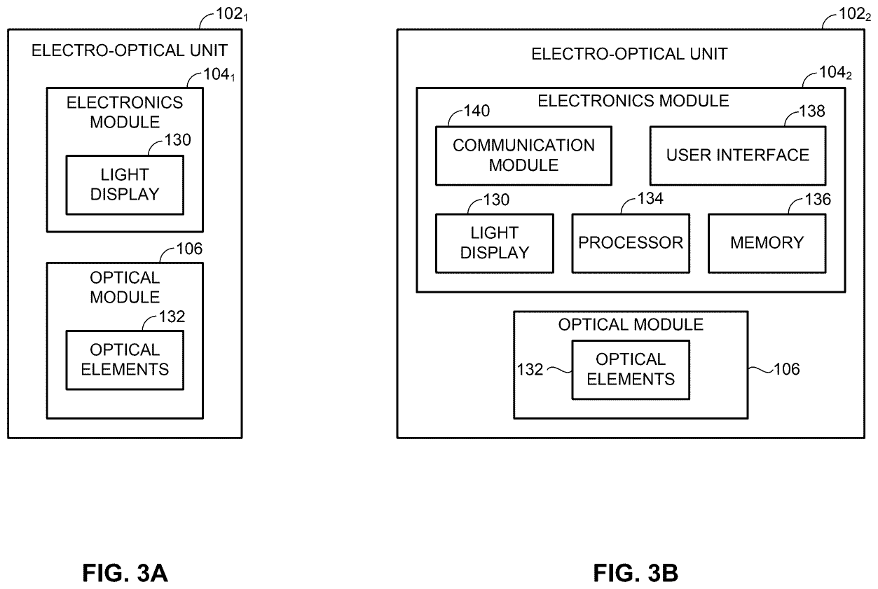

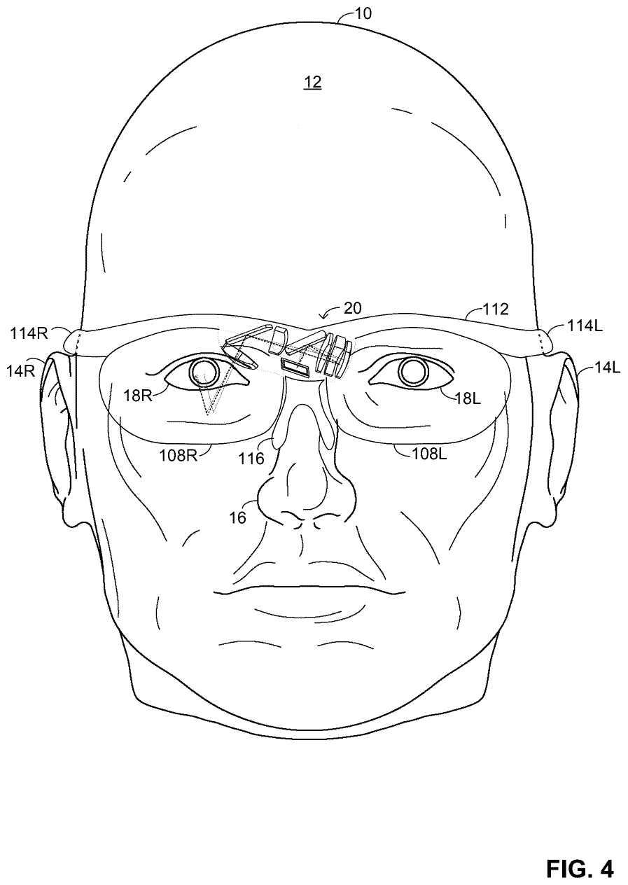

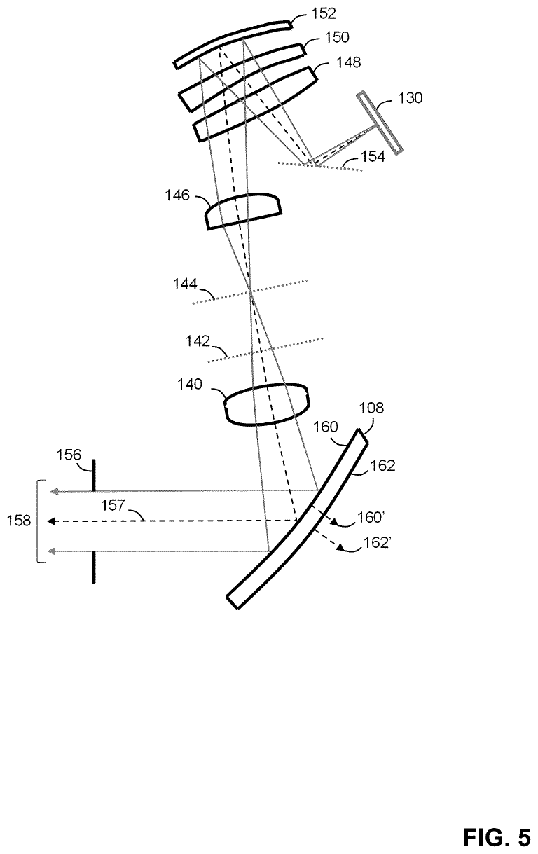

Reference is now further made to FIGS. 3A, 3B, 4, and 5. FIG. 3A is a schematic block diagram of a basic configuration of the electro-optical unit of OST NED system, constructed and operative in accordance with the embodiment of the disclosed technique. FIG. 3B is a schematic block diagram of an accessorized configuration of the electro-optical unit of OST NED system, constructed and operative in accordance with the embodiment of the disclosed technique. FIG. 4 is a schematic illustration showing OST NED system of FIGS. 1 and 2 in a mounted configuration on a user. FIG. 5 is a schematic diagram of a light path through an optical module of electro-optical unit of OST, constructed and operative in accordance with the embodiment of the disclosed technique. FIG. 3A illustrates a basic configuration of electro-optical unit 102 of OST NED system 100, denoted by 102₁. Electro-optical unit 102₁ includes an electronics module 104₁, and optical module 106. Electronics module 104₁ includes a light display 130. Optical module 106 includes optical elements, collectively referenced 132 that are particularized in greater detail in FIG. 5. Light display 130 of electronics module 104₁ along with optical elements 132 of optical module 106 form a light projector that is configured and operative to produce, irradiate, and project a light beam image (not shown) onto at least one partially transmissive partially reflective lens 108 (i.e., at least one of 108R and 108L). Optical elements 132 are configured and operative to convey and project the image produced by light display 130 onto partially transmissive partially reflective lens 108, as will be described in greater detail below in conjunction with FIGS. 4 and 5.

FIG. 3B illustrates an accessorized configuration of electro-optical unit 102 of OST NED system 100, denoted by 1022, having additional components with respect to the basic configuration of electro-optical unit configuration 1021, shown in FIG. 3A. Electro-optical unit 102 includes an electronics module 1042, and optical module 106 (i.e., identical to that in FIG. 3A). According to the accessorized configuration, electronics module 1042 includes light display 130 (i.e., identical to that in FIG. 3A), and may optionally include at least one of a processor 134, a memory device 136, a user interface 138, and a communication module 140. Processor 134 is configured and operative to be communicatively coupled (i.e., wired, wirelessly thereby enabling to at least one of send and receive signals and data) to light display 130, memory device 136, user interface 138, and communication module 140 (interconnections not shown in FIG. 3B). Electro-optical unit 1022 is electrically powered by a power source (not shown). The power source may be embodied in the form of at least one battery that may be housed in user attachment section 112, such as in stem portions 114R and 114L. Alternatively or additionally, solar arrays (not shown) may be integrated with frame 112 and/or with partially transmissive partially reflective lens 108 (e.g., by employing transparent solar arrays, known in the art (e.g., solar window technology)). User interface 138 is configured to communicate data with processor 134, and to perform the following functions: receive an input from the user, transmit an output to the user, present information to the user, enable interaction with the user, and communicate with the user.

With reference to FIG. 4, frame 112 (FIGS. 1 and 2) enables detachable mounting of OST NED 100 system to a head 12 of user 10. Particularly, FIG. 4 shows OST NED system 100 in a mounted configuration on head 12 of user 10 (i.e., being worn). Temples 114R, 114L, and nosepiece 116 are constructed and operative to support OST NED system 100, on the ears 14R, 14L and nose 16 (respectively) of user 10 (FIG. 4). Partially transmissive partially reflective lenses 108R and 108L are coupled with frame 112, and each configured to be facing eyes 18R and 18L (respectively) of user 10 wearing OST NED system 100. Specifically, lens 108R is configured to be facing a right eye 18R, and lens 108L is configured to be facing a left eye 18L of user 10. When OST NED system 100 is in a mounted configuration on user 10, user attachment section 112 is configured to enable electro-optical unit 102 including housing 110 to be positioned within a glabellar region 20 of user's head 12, as shown in FIG. 4. Glabellar region 20 is defined herein as an area covering the glabella (also known as the mesophryon), which, in humans, is an anatomical area between the eyebrows and above the nose 16.

Optical elements (components) 132 (FIGS. 3A and 3B) of optical module 106 as shown in FIG. 4 are further described in greater detail in conjunction with FIG. 5 that illustrates a schematic diagram of a light path through optical module 106 of electro-optical unit 102 of OST NED 100. Electro-optical unit 102 (i.e., 102₁ and 102₂) includes light display 130, and optical elements 132 of optical module 106 that include a first lens 140 (FIG. 5), a reflector 142, a second lens 146, a third lens 148, a fourth lens 150, a curved mirror 152, and a partially transmissive partially reflective element 154. Further shown in FIG. 5 is partially transmissive partially reflective lens 108, and aperture 156 (herein denoted interchangeably as “light display exit pupil”), and an intermediate image (not shown) that forms at an intermediate image plane 144 between first lens 140 and second lens 146. In general, each partially transmissive partially reflective lens 108 includes an inner surface 160 (herein interchangeably denoted “user-facing surface”), and an outer surface 162 (herein interchangeably denoted “outward-facing surface”) Inner surface 160 is characterized by an inner surface radius of curvature rᵢ exhibiting a first optical power Pᵢ. Outer surface 162 is characterized by an outer surface radius of curvature rₒ exhibiting a first optical power Pₒ. In a mounted configuration of OST NED system 100 on user 10, inner surface 160 is configured to be facing an eye 14(R, L) of user 10 and outer surface 162 is configured to be facing an outward scene directed away from user 10. Partially transmissive partially reflective lens 108 is configured to at least partially transmit incoming light of an outward scene, impinging on outer surface 162 and exiting from inner surface 160 toward eye 14(R, L) of user 10.

Optical elements 132 of optical module 106, light display 130 of electronics module 104, and partially transmissive partially reflective lens 108 form an optical arrangement (herein interchangeably “optical configuration”) allowing light rays to propagate as illustrated in FIGS. 4 and 5. The optical configuration enables light display 130 to project a light beam image (not shown) onto partially transmissive partially reflective lens 108 (i.e., at least one of 108R and 108L) along an optical path represented by light rays 158 and the optical axis 157. Specifically, light display 130 is configured and operative to produce and irradiate the light beam image (i.e., light encoded data defined as light containing data) that propagates along the optical path described as follows. Light display 130 produces the light beam that impinges onto first main reflector 154, which in turn is configured to reflect the light beam toward third lens 148, which in turn is configured to refract the light beam and convey it toward fourth lens 150, which in turn is configured to refract the light beam and convey it toward curved mirror 152. Curved mirror 152 is configured to reflect the light beam back toward fourth lens 150, which in turn is configured to refract the light beam again in reverse order and convey it toward third lens 148. Third lens 148 is configured to refract the light beam and convey it toward second lens 146, which in turn is configured to refract the light beam and convey it toward second auxiliary reflector 142. Second auxiliary reflector 142 is configured to fold the light beam (not shown) and reflect it toward first auxiliary reflector, which in turn is configured to fold the light beam (not shown) and reflect it toward first lens 140. First lens 140 is configured to refract the light beam and convey it toward partially transmissive partially reflective lens 108. Partially transmissive partially reflective lens 108 is configured to reflect the light beam toward at least one eye 18R, 18L of user 10.

Inner surface 160 of partially transmissive partially reflective lens 108, which is characterized by inner surface radius of curvature rᵢ exhibiting a tilt angle 160′ with respect to the optical axis, is configured to reflect the light beam emerging from first lens 140 to the eye of the user.

The optical path of the light beams (i.e., exemplified as rays 158 in FIG. 5) originate from light display 130, reflect off partially transmissive partially reflective element 154, pass through lenses 148, 150, reflect from curved mirror 152, pass through (i.e., refract by) lenses 150 and 148 again (i.e., and in reverse order), then pass through lenses 146, and 140, and in due course, reflect from inner surface 160 of partially transmissive partially reflective lens 108 toward an eye (18R, 18L) of user 10. Intermediate image forms along the optical path between first lens 140 and second lens 146, and particularly between auxiliary reflector 142 and second lens 146. Specifically, second lens 146 is configured and operative to be positioned along the optical path and have optical characteristics (e.g., optical power) that allows intermediate image to form at intermediate image plane 144 located perpendicularly along the optical path between itself (second lens 146) and first lens 140. Auxiliary reflector 142 (e.g., fold mirror) facilitates in the minimization of the spatial dimensions of electro-optical unit 102 so that it may substantially meet the spatial constraints of being located substantially at glabellar region 20 of user 10.

In accordance with the disclosed technique, OST NED system 100 is configured and operative to generate and to project light encoded data (i.e., light containing data) in the form of a light representation (e.g., an image, graphical information, symbology, etc.) onto partially transmissive partially reflective lens 108, the latter of which is configured and operative concurrently, to at least partially reflect the light (i.e., which is encoded with data or that contains information) toward the eyes (18R, 18L) of user 10 (FIG. 4, shown as an example for right eye 18R) who wears the system, as well as to at least partially transmit incoming light from a scene in the user's field of regard (i.e., outward-facing environment with respect to the user). Partially transmissive partially reflective lens 108 essentially acts as an optically collimated transparent (or translucent) display enabling the overlay of images and other data projected onto its surfaces (i.e., a combiner), without obstructing a scene viewed by the user.

In accordance with the disclosed technique, OST NED system 100 is configured and operative to generate and to project light encoded data (i.e., light containing data) in the form of a light representation (e.g., an image, graphical information). In the basic configuration shown in FIG. 3A, light display 130 is configured to receive data (e.g., image information from an external source (not shown)) and to generate, irradiate, and project light encoded data in the form of a light representation and to convey the light encoded data toward optical elements 132 of electro-optical unit 102. Alternatively, light display 130 incorporates an internal memory unit (not shown) configured and operative to store data (e.g., image information), which light display 130 uses to generate the light encoded data.

In the accessorized configuration shown in FIG. 3B, processor 134 (FIG. 3B) is configured and operative to produce, process, and modify, data (not shown), for example in the form of electrical signals that convey image data and/or graphical representation data and to provide (communicate) this data to light display 130. Memory device 136 is configured and operative to store this image data for manipulation (e.g., by processing and modification) and for retrieval (e.g., by processor, by light display 130). Memory device 136 is generally embodied in the form of non-volatile memory (e.g., read-only memory (ROM), flash memory, magnetic storage devices (e.g., hard disks), ferroelectric read-only memory (F-RAM), optical memory (e.g., optical discs), etc.) as well as volatile memory (e.g., RAM).

Light display 130 is configured and operative to receive data, generate light encoded data based on the data received therefrom, irradiate and project the light encoded data toward partially transmissive partially reflective element 154. The terms “light encoded data” and “light encoded information” used interchangeably herein, generally refer to light that is encoded with data, and more specifically, to light that contains information that is exhibited in at least one domain, such as in the spectral domain (i.e., wavelengths-colors), in the spatial domain (e.g., in one or multi-dimensions, such as a one-dimensional (1-D) image (e.g., a point, or pixel), a two-dimensional (2-D) image, a three-dimensional (3-D) image), in the temporal domain (e.g., changing-frame rate), in the polarization domain (e.g., by using light polarization encoding techniques), and the like. For example, in case where the encoded data is 2-D color video, the light encoded data that is generated and projected exhibits change in the temporal domain (i.e., as a succession of image frames), in the spatial domain (i.e., as changes in the image space-as in the case of moving objects within the image frames), in the color domain (i.e., representing the different colors of objects in the images), as well in the light intensity domain (i.e., representing, for example the luminance).

Particularly, light display 130 receives data (e.g., externally, from an internal memory device, from processor 134), and generates a collimated light beam (encoded with data) shown representationally in FIG. 5 as a plurality of light rays emanating from light display 130 toward partially transmissive partially reflective element 154. Partially transmissive partially reflective element 154 reflects the light beam toward lens 148, which is typically a convex-concave (meniscus) lens, which in turn refracts this light beam and relays the refracted light beam toward lens 150, the latter of which is optically aligned with respect to lens 148. Lens 150 refracts the refracted light beam relayed from lens 148 and directs the beam toward curved mirror 152, the latter of which is optically aligned with the former. Lens 150 is typically a concave-convex (meniscus) lens, and curved mirror 152 is typically a concave spherical (front surface or alternatively, a rear surface) mirror. Alternatively, mirror 152 may exhibit curvatures other than spherical, such as aspherical, hyberbolic, elliptical, parabolic, toroidal, and the like. The optical elements group (combination) which includes lens 148, lens 150, and curved mirror 152 is constructed and optically arranged to correct aberrations such as astigmatism and distortion that is caused by the optical characteristics such as the curvature of partially transmissive partially reflective lens 108 and its tilt angle with respect to the optical axis. The refracted light beam arriving from lens 150 impinges curved mirror 152 such that the consequent reflected light beam is directed to pass (again) through lenses 150 and 148, thereby forming an angle with respect to the incoming refracted light beam. The combination of curved mirror 152 and lenses 148 and 150 allow the incoming light beam generated and irradiated from light display 130 to pass (refract) twice through lenses 148 and 150, thereby enabling pre-emptive correction of astigmatism and distortion caused when the light beam reflects off partially transmissive partially reflective lens 108. Generally, in oblique astigmatism, off-axis rays of light from radial and tangential lines in an object plane focus at different distances in the image space. The utilization of lens 148 with lens 150 in the optical arrangement functions to increase the optical power of the optical system by decreasing the overall focal length of optical elements and in effect, enabling a reduction of the physical dimensions of electro-optical unit 102, as well as further enabling fine-tuning for minimizing astigmatism, distortion and chromatic aberrations.

Second lens 146 receives the refracted light beam from third and fourth lenses 148 and 150, refracts and relays the light beam toward first lens 140 in optical module 106. Third and fourth lenses 148 and 150 are typically of biconvex type (although other types may be used, e.g., plano-convex). As mentioned, second lens 146 is optically configured and operative to form intermediate image (not shown) at an intermediate image plane 144 located at a position along an optical path between first lens 140 and second lens 146. Further located at a position along this optical path between first and second lenses 140 and 146 is positioned auxiliary reflector 142 (e.g., a planar folding mirror), which is optically configured and operative to reflect light beams from second lens 146 toward first lens 140 and is employed to bend the optical path into a particular spatial configuration (e.g., required at least for minimizing the spatial dimensions of housing 110 (FIG. 2) of electro-optical unit 102). Minimization of spatial dimensions that is at least partially enabled by bending the light beams of the optical path facilitate in meeting the spatial constraints of housing 110 being located substantially at glabellar region 20 of user 10. The formation of an intermediate image facilitates in the magnification of on outputted generated image, as well as in the widening of the field of view (FOV) that is presented to the user. The formed intermediate image is an aberration-compensated image (or aberration-corrected image) owing to the optical arrangement (and optical characteristics) of curved mirror 152 and lenses 148 and 150. This aberration-compensated formed intermediate image forestalls aberrations produced, at least partially, by the curvature of partially transmissive partially reflective lens 108, such that light rays incident upon and reflecting therefrom are aberration-corrected.

Partially transmissive partially reflective lens 108 is constructed and operative to partially reflect the light beam impinging thereon from light display 130 (i.e., an image source), and partially transmit incoming light from an outward translucent view in the ambient environment (i.e., a scene). Partially transmissive partially reflective lens 108 effectively functions as a light combiner, such that light from the image source and light from the outward scene are combined to form a combined image (not shown) that is directed and provided to at least one eye of the user. Both partially transmissive partially reflective lenses 108L and 108R for both the user's left and right eyes 18L and 18R are adapted to match the interpupillary distance (IPD) of that user. As such, partially transmissive partially reflective lens 108 is typically constructed from rigid, durable, lens-grade materials such as glass (e.g., optical crown glass), polycarbonate, and the like, as well as at least one reflective optical coating layer whose thickness (at least to some degree) determines its reflective characteristics. The ratio of reflection to transmission, which is typically dependent on the wavelength of light incident on partially transmissive partially reflective lens 108, may vary. Typical reflection percentages typically lie between 10-40%; whereas typical total transmission percentages lie between 8-80%. Any ratio derived from these percentages is viable. In particular, there may be more than one reflection to transmission ratios of partially transmissive partially reflective lens 108 (which may be different or the same). One reflection to transmission ratio is associated with light impinging on inner surface 160 (produced by light display 130), the other associated with light impinging on outer surface 162. According to one realization, the reflectivity of outer surface 162 may be greater than the reflectivity of inner surface 160. Other, different realizations may be possible, for example, where outer surface 162 (i.e., and/or at least part of partially transmissive partially reflective lens 108) may be embedded with silver halide (silver salts) in microcrystalline form that endow photochromic properties to the lens. The following features and options may apply differently or equally to each partially transmissive partially reflective lens 108R and 108L. Optionally, an antireflection coating may also be applied to outer surface 162. Further optionally, an anti-abrasion coating may also be applied to partially transmissive partially reflective lens 108. Further optionally, an anti-fog coating may be applied to partially transmissive partially reflective lens 108. Further optionally, partially transmissive partially reflective lens 108 may be coated and/or incorporate light-polarized material, which generally enhances the contrast of an image viewed through the lens (e.g., especially noticeable in snow covered environments). Partially transmissive partially reflective lens 108 may employ shatter resistant (“shatterproof”) materials (e.g., polyvinyl butyral (PVB), ethylene-vinyl acetate (EVA), thermoplastic polyurethane (TPU), polyethylene terephthalate (PET), polycarbonate (PC), combinations thereof, etc.) in its construction and/or manufacture such that it exhibits shatterproof and impact-resistant qualities. Moreover, partially transmissive partially reflective lens 108 complies with known safety standards of eyewear in general, and eyewear (e.g., sunglasses) used for sporting applications, in particular. Partially transmissive partially reflective lens 108 reflects the light beam from first lens 140, passing through light display exit pupil 156 and toward the eye of the user.

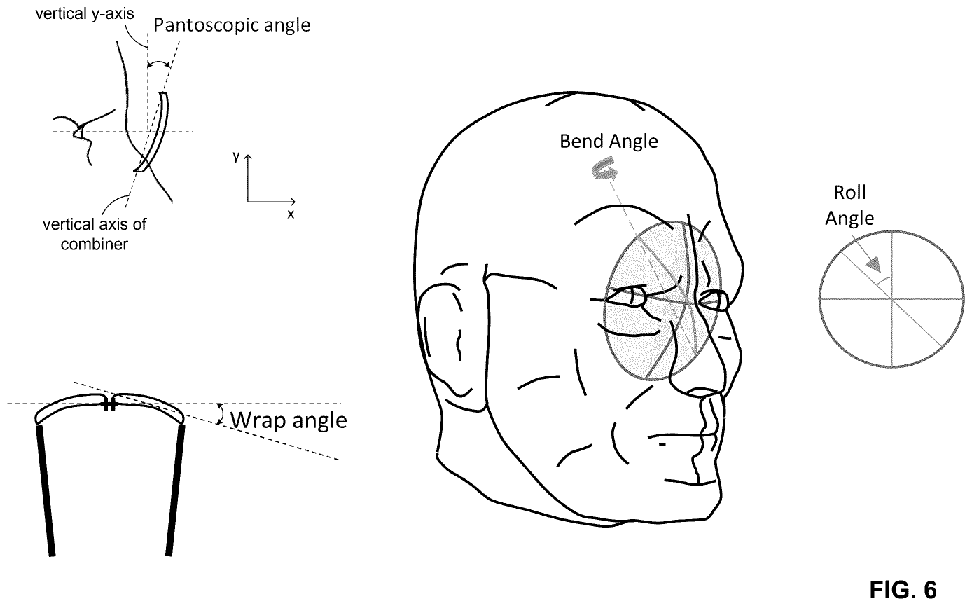

Reference is now further made to FIG. 6, which is a schematic illustration showing a pantoscopic angle, a wrap angle, a bend angle, and a roll angle according to the principles of the disclosed technique. The pantoscopic angle is defined as an angle between a plane of combiner 108 and the vertical y-axis, as indicated in FIG. 6. Mathematically, the pantoscopic angle is defined approximately as: cos(Roll)*Bend. More precisely, the pantoscopic angle is defined as: tan⁻¹(tanα*cosγ) where α denotes the bend angle, and γ denotes the roll angle. FIG. 6 shows a side partial view of the user, and combiner 108, where the vertical dotted line indicates the vertical axis (y-axis) and a pantoscopic angle from a plane of combiner 108 to the vertical axis. FIG. 6 further shows a wrap angle which is defined as half the angle of frame 112 to a straight line, and approximately as: sin(Roll)*Bend. More precisely, the wrap angle is mathematically defined as: tan⁻¹(tanα*sinγ) where α denotes the bend angle, and γ denotes the roll angle. The roll angle as shown in FIG. 6 is defined as the projected exit aperture angle, as illustrated.

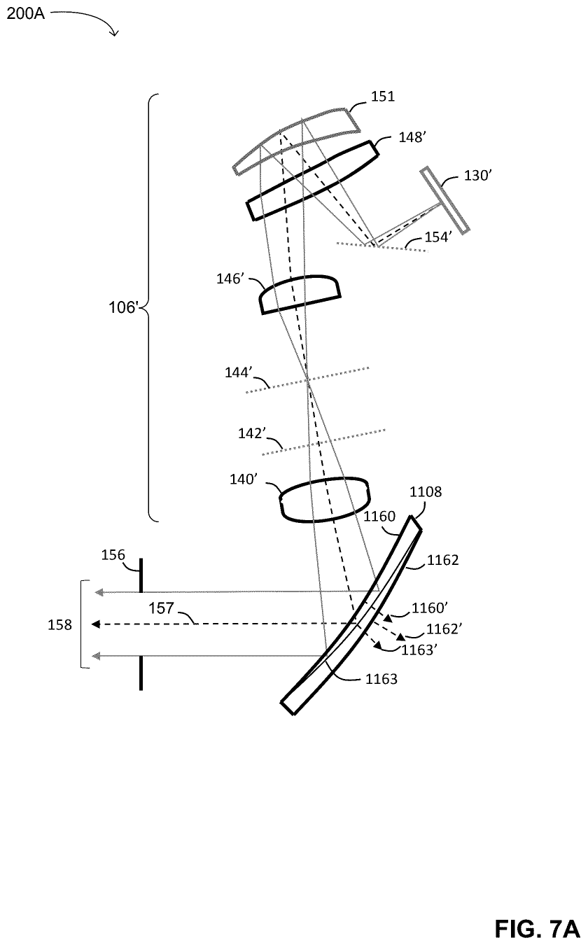

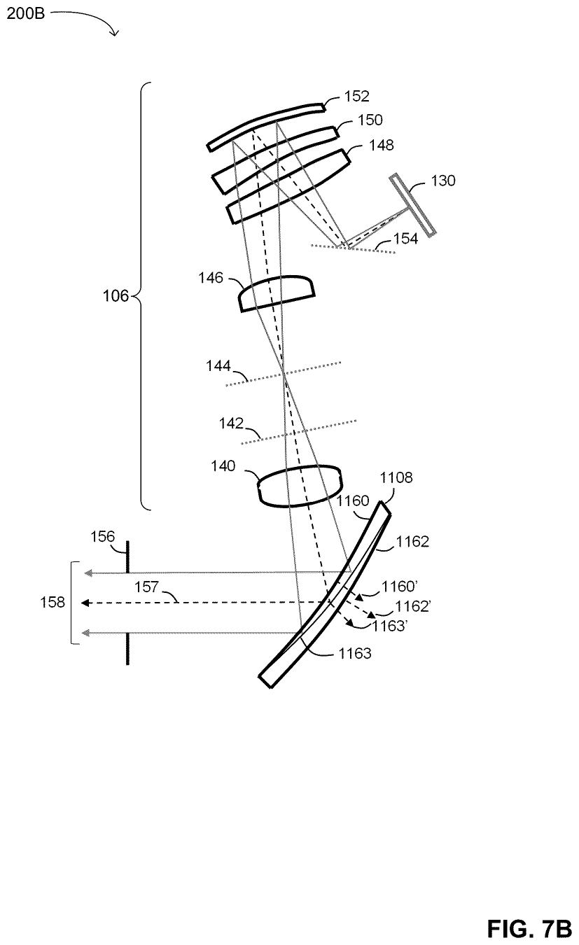

Reference is now made to FIG. 7A, which is an illustration showing a schematic diagram of a light path through a glabellar-region positioned electro-optical subsystem in an optical see-through (OST) near-eye display (NED) off-axis system for improving a pantoscopic angle, generally referenced 200A, constructed and operative in accordance with an embodiment of the disclosed technique. All elements of system 200A are the same as those respective elements of system 100, except for those elements referenced, and numbered differently. System 200A includes an electro-optical unit 102′ (not number referenced in FIG. 7A), which generally includes an electronics module 104′ (not number referenced in FIG. 7A, and similar to electronics module 104 of FIG. 2 and electronics module 104₁ of FIG. 3A and electronics module 104₂ of FIG. 3B.), and an optical module 106′. Electronics module 104′ of Electro-optical unit 102′ includes a light display 130′. Optical module 106 includes a plurality of optical elements 132′. Specifically, optical module 106′ includes a first lens 140′, a reflector 142′, a second lens 146′, a third lens 148′, and a reflective lens 151 (interchangeably denoted herein “fourth reflective lens”) embodied herein as an off-axis Mangin mirror. FIG. 7A further shows an aperture 156 (herein denoted interchangeably as “light display exit pupil”), an intermediate image (not shown) that forms at an intermediate image plane 144′ between first lens 140′ and second lens 146′. Partially transmissive partially reflective lens 1108 includes an inner surface 1106 (herein denoted interchangeably “user facing surface”), an outer surface 1162 (herein denoted interchangeably “outward-facing surface”), and an intermediate reflective surface 1163 that is embedded and enclosed within the internal volume of partially transmissive partially reflective lens 1108. Inner surface 1160 is characterized by an inner surface radius of curvature rᵢ exhibiting a first optical power Pᵢ. Outer surface 1162 is characterized by an outer surface radius of curvature rₒ exhibiting a first optical power Pₒ. Intermediate reflective surface 1163 is characterized by an intermediate reflective surface radius of curvature rᵣ exhibiting a reflective surface optical power Pᵣ.

Intermediate reflective surface 1163 is curved (concave) and extends from one (top) extremity of outer surface 1162 and to another (bottom) extremity of inner surface 1160, within the internal volume of the refractive material of partially transmissive partially reflective lens 1108, as shown in FIG. 7A. Intermediate reflective surface 1163, which is concave is structured into the bulk volume of partially transmissive partially reflective lens 1108 and positioned in-between inner surface 1160 (“inner concave surface”) and outer surface 1162 (“outer concave surface”). Intermediate reflective surface 1163 is thus an intermediate concave surface that is positioned in-between the two concave surfaces (i.e., inner and outer concave surfaces 1160 and 1162, respectively), traversing obliquely and not at the mid-distance therebetween along the entire length of intermediate reflective surface 1163. Intermediate reflective surface 1163 is coated with a partially transparent partially reflective (e.g., semitransparent reflective) material (not shown) that is configured and operative to reflect light rays impinging thereon. Alternatively, intermediate reflective surface 1163 is coated with a partially transparent partially reflective material on a portion of the surface. Intermediate reflective surface 1163 is configured to reflect a light beam 158 that emerges from lens 140′. The light beam then partially refracts via partially transmissive partially reflective lens 1108, impinges and reflects from intermediate reflective surface 1163, refracts again through partially transmissive partially reflective lens 1108, and emerges along optical axis 157 toward the eye(s) of the user.

The light-projected optical path of system 200A starts from light display 130′ that electronically receives data (e.g., externally, from an internal memory device, from processor 134 —FIGS. 3A and 3B), and generates a collimated light beam (e.g., encoded with data) shown representationally in FIG. 7A as a plurality of light rays emanating from light display 130′ along an optical path ultimately reaching the eye of the user as light beam 158, along optical axis 157. Reflective element 154′ reflects the light beam toward lens 148′, which in turn refracts this light beam and relays the refracted light beam toward reflective lens 151, the latter of which is an off-axis Mangin mirror that is optically aligned with respect to lens 148′. Mangin mirror 151 is constructed from two main portions: (1) a concave lens portion (having a front surface (interchangeably denoted herein “first surface”) and a rear surface (interchangeably denoted herein “second surface”)); and (2) a reflective concave surface (disposed along the rear/second surface). The front (first) and rear (second) surfaces of Mangin mirror 151 have different radii of curvatures with respect to each other. According to one optical configuration, the first surface is tilted with respect to the second surface of Mangin mirror 151. According to another, optical configuration the first surface and second surface of Mangin mirror 151 are centered or coaxial (i.e., not tilted with respect to each other). The incoming light beam passes through Mangin mirror 151 via the first surface, the concave lens portion refracts the light beam, then the reflective concave surface portion reflects the light beam back to the concave lens portion, which in turn refracts the light beam again (a second time) through the lens portion, where now this lens portion exhibits a convex shape with respect to the propagating direction of the reflected outgoing light rays. Reflective lens 151 (i.e., Mangin mirror) is constructed and operative to refract and reflect the light beam back to lens 148′ such that the light beam refracts twice through lens 148′. This enables to pre-emptively correct astigmatism and distortion caused when the light beam reflects off partially transmissive partially reflective lens 1108. Effectively, the light beam refracts twice through lens 148, and twice through reflective lens 151, as diagrammatically shown in FIG. 7A. The lens portion of Mangin mirror 151 is constructed from a material that generally has a refractive index different from the refractive index of the air.

Off-axis Mangin mirror 151 is constructed, designed, and operative to compensate for residually imposed optical aberrations in the structure of partially transmissive partially reflective lens 1108 in comparison to that of partially transmissive partially reflective lens 108 of FIG. 5. The different optical configurations of FIG. 7A with respect to that of FIG. 5 results in a change in their optical paths, in which a light beam that propagates from lens 140 (FIG. 5) and lens 140′ (FIG. 7A) toward the user's eye is reflected from concave inner surface 160 (FIG. 5) and concave intermediate reflective surface 1163 (FIG. 7A). Off-axis Mangin mirror 151 is further constructed, designed, and operative to compensate for optical power, astigmatism, and chromatic aberrations. Specifically, the construction of Mangin mirror 151 involves the optimization of the concave shape of the first surface, the concave shape of the second surface, the decenter and tilt of the second surface with respect to the first surface, the selection of the material(s) of the lens portion (and the reflective portion), as well as the geometry (e.g., thickness). Typically, the shape of the first surface and the second surface of Mangin mirror 151 are constructed from a spherical, toroid, aspherical, or free-form shape. The decenter and tilt characteristics of the second surface in relation to the first surface, allows for an additional optimization to compensate for the residual astigmatism aberrations caused by the off-axis optical configuration, due to light beam refraction by the refractive material of partially transmissive partially reflective lens 1108. The material composition of Mangin mirror 151 allows for an additional optimization in order to compensate for chromatic aberrations that may result by the optical elements of the system in general, and by material composition of partially transmissive partially reflective lens 1108, in particular.

Lens 148′, which refracts the light beam twice, following the second refraction, transmits the light beam toward lens 146′, which in turn, receives the refracted light beam and refracts and relays the light beam toward lens 140′ in optical module 106′. Lens 146′ is optically configured and operative to form an intermediate image (not shown) at an intermediate image plane 144′ located at a position along an optical path between lens 140′ and lens 146′. Further located at a position along this optical path between first and second lenses 140′ and 146′ is positioned an auxiliary reflector 142′ (e.g., a planar folding mirror), which is optically configured and operative to reflect light beams from second lens 146′ toward first lens 140′ and is employed to bend the optical path into a particular spatial configuration (e.g., required at least for minimizing the spatial dimensions of housing 110 (FIG. 2) of the electro-optical subsystem).

Lens 140′ refracts the light beam and conveys it toward partially transmissive partially reflective lens 1108. Specifically, partially transmissive partially reflective lens 1108 has an inner surface 1160 exhibiting an inner radius of curvature with a tit angle of 1160′, an outer surface 1162 exhibiting an outer radius of curvature with a tilt angle 1162′, and an intermediate reflective surface 1163, located in between the inner and outer surfaces, having a reflective surface radius of curvature with a tilt angle 1163′. Intermediate reflective surface 1163 is positioned and integrated within partially transmissive partially transmissive lens 1108. Light display 130′ projects a light beam (via optical elements of optical module 106′) onto partially transmissive partially reflective lens 1108, which passes through inner surface 1160, propagates through the refractive material of partially transmissive partially reflective lens 1108, reflects back from intermediate reflective surface 1163, propagates again through the refractive material of partially transmissive partially reflective lens 1108, passes through inner surface 1160 and propagates through the air toward the eye of the user along optical axis 157. Inner surface 1160 can be coated with an anti-reflective coating to minimize residual reflections allowing for maximal optical efficiency, as the light beam refracts back and forth through inner surface 1160. Partially transmissive partially reflective lens 1108 and a mirror coating of the intermediate reflective surface 1163 reflects a portion of the light beam and refracts another portion of the light beam, which in turn passes through the reflective surface, propagating through the refractive material of partially transmissive partially reflectance lens 1108 reaching outer surface 1162 and disperses into the surrounding medium (e.g., air). Outer surface 1162 can be coated with an anti-reflective coating to minimize residual reflections, which may cause residual optical aberrations such as ghosting and stray light.

Reference is now made to FIG. 7B, which is an illustration showing a schematic diagram of a light path through a glabellar-region positioned electro-optical subsystem in an optical see-through (OST) near-eye display (NED) off-axis system for improving a pantoscopic angle, generally referenced 200B, constructed and operative in accordance with another embodiment of the disclosed technique. All elements of system 200B are the same as those respective elements of systems 100 and 200A, except for those elements referenced, and numbered differently. System 200B generally combines the elements of system 100 (FIG. 5) and system 200A (FIG. 7A). System 200B includes electro-optical unit 102 (not number referenced in FIG. 7B), which in turn includes an electronics module 104 (not number referenced in FIG. 7B, see FIG. 2), and an optical module 106. Electronics module 104 of electro-optical unit 102 includes a light display 130. Optical module 106 includes a plurality of optical elements 132. Optical module 106 includes a first lens 140, a reflector 142, a second lens 146, a third lens 148, a fourth lens 150, and a curved mirror 152. FIG. 7B further shows an aperture 156, an intermediate image (not shown) that forms at an intermediate image plane 144 between first lens 140 and second lens 146. Partially transmissive partially reflective lens 1108 includes an inner surface 1106 (herein denoted interchangeably “user facing surface”), an outer surface 1162 (herein denoted interchangeably “outward-facing surface”), and an intermediate reflective surface 1163 that is enclosed and embedded within the internal volume of the refractive material of partially transmissive partially reflective lens 1108. Inner surface 1160 is characterized by an inner surface radius of curvature rᵢ exhibiting a first optical power Pᵢ, and tilt angle 1160′. Outer surface 1162 is characterized by an outer surface radius of curvature rₒ exhibiting a first optical power Pₒ, and a tilt angle 1162′. Intermediate reflective surface 1163 is characterized by a reflective surface radius of curvature rᵣ exhibiting a reflective surface optical power Pᵣ, and a tilt angle 1163′.

Intermediate reflective surface 1163 is curved (concave) and extends from one (top) extremity of outer surface 1162 and to another (bottom) extremity of inner surface 1160, within the internal volume of partially transmissive partially reflective lens 1108, as shown in FIG. 7B (similarly to that shown in FIG. 7A). Intermediate reflective surface 1163 is constructed into the bulk volume of partially transmissive partially reflective lens 1108 and positioned in-between inner surface 1160 (“inner concave surface”) and outer surface 1162 (“outer concave surface”). Intermediate reflective surface 1163 is coated with a semitransparent reflective material (not shown) that is configured and operative to reflect light rays impinging thereon. Specifically, intermediate reflective surface 1163 is configured to reflect a light beam that emerges from lens 140. The light beam partially refracts via partially transmissive partially reflective lens 1108, impinges and reflects from intermediate reflective surface 1163, refracts again through partially transmissive partially reflective lens 1108, and emerges along optical axis 157 toward the eye(s) of the user.

The light-projected optical path of system 200B starts from light display 130 that generates a light beam (e.g., encoded with data) that emanates from light display 130 along an optical path that is destined to reach the eye of the user. Reflective element 154 reflects the light beam toward lens 148, which in turn refracts the light beam toward lens 150, which in turn refracts the light beam and relays it to curved mirror 152, which in turn reflects the light beam back to lens 150 in reverse order such that lens 150 refracts the light beam again and conveys it to lens 148. Lens 148 refracts the light beam a second time and conveys the light beam toward lens 146. FIG. 7B shows lenses 148 and 150 refracting the light beam twice, in generally opposing directions, after reversing directions by reflection off curved mirror 152. Lens 146 is optically configured and operative to form an intermediate image (not shown) at an intermediate image plane 144 located at a position along an optical path between lens 140 and lens 146. Further located at a position along this optical path between first and second lenses 140 and 146 is positioned an auxiliary reflector 142 (e.g., a planar folding mirror), which is optically configured and operative to reflect light beams from second lens 146 toward first lens 140 and is employed to bend the optical path into a particular spatial configuration (e.g., required at least for minimizing the spatial dimensions of housing 110 (FIG. 2) of the electro-optical subsystem). Lens 140 refracts the light beam and conveys it toward partially transmissive partially reflective lens 1108.

Intermediate reflective surface 1163 is positioned and integrated within partially transmissive partially transmissive lens 1108. Intermediate reflective surface 1163 is configured to reflect the light beams from the electro-optical subsystem to the eye of the user. Specifically, light display 130 projects the light beam (via optical elements of optical module 106) onto partially transmissive partially reflective lens 1108, such that the light beam passes through inner surface 1160, propagates through the refractive material of partially transmissive partially reflective lens 1108, reflects back from intermediate reflective surface 1163, propagates again through the refractive material of partially transmissive partially reflective lens 1108, passes through inner surface 1160 and propagates through the air toward the eye of the user along optical axis 157. Preferably, inner surface 1160 is coated with an anti-reflective coating to minimize residual reflections so to allow for maximal optical efficiency as the light beam refracts back and forth through inner surface 1160. Partially transmissive partially reflective lens 1108 and a mirror surface of the intermediate reflective surface 1163 reflects a portion of the light beam and refracts another portion of the light beam, which in turn passes through the intermediate reflective surface propagating through the refractive material of partially transmissive partially reflectance lens 1108 reaching outer surface 1162 and disperses into the surrounding medium (e.g., air). Outer surface 1162 can be coated with an anti-reflective coating to minimize residual reflections, which may cause residual optical aberrations such as ghosting and stray light.

The optical design of system 200B takes into account that the optical path of the light beam propagating from lens 140 toward the eye of the user, passes through free-space (e.g., air) then through the refractive material of partially transmissive partially reflective lens 1108, and then through free-space again. The refractive index of partially transmissive partially reflective lens 1108 (e.g., 1.59, 1.61, 1.67) is different than from air (~1 at sea level under standard conditions). Furthermore, the Abbe number of partially transmissive partially reflective lens 1108 has an Abbe number (V-number) different than that of air. The Abbe number measures the material's (partially transmissive partially reflective lens 1108) chromatic dispersion.

With reference to both FIGS. 7A and 7B, the incorporation of Mangin mirror 151 in system 200A allows to eliminate lens 150 and curved mirror 152 from the optical path thus improving system 200A with respect to system 200B, at least by shortening the entire optical path, leading to the reduction in size of electro-optical unit 102′ (FIG. 7A) with respect to electro-optical unit 102 (FIG. 7B). Moreover, the use of Mangin mirror 151 in the optical configuration of system 200A convers the aforementioned advantages associated with the characteristics of the Mangin mirror, as detailed herein above. It is noted that the image produced by system 200B (FIG. 7B) is affected by astigmatism, which does not occur in system 200A (FIG. 7A), that latter of which is configured to correct for astigmatism (or residual astigmatism).

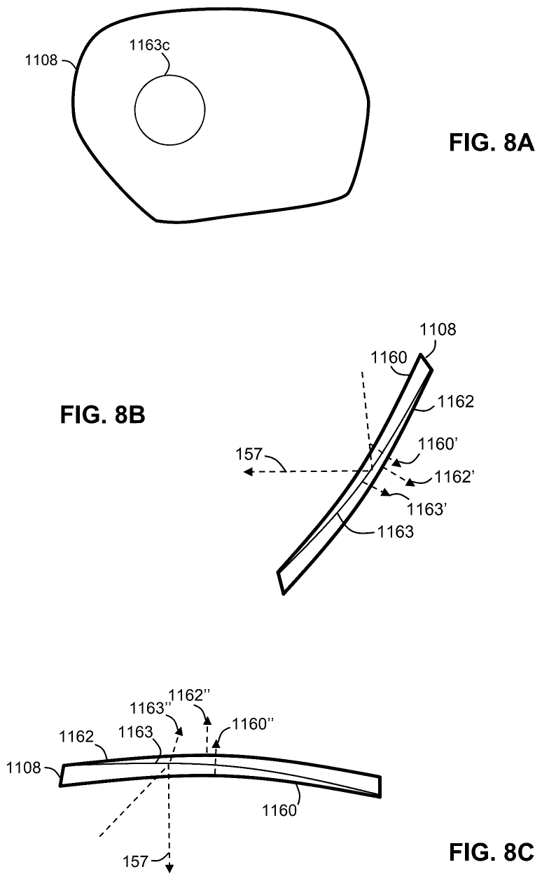

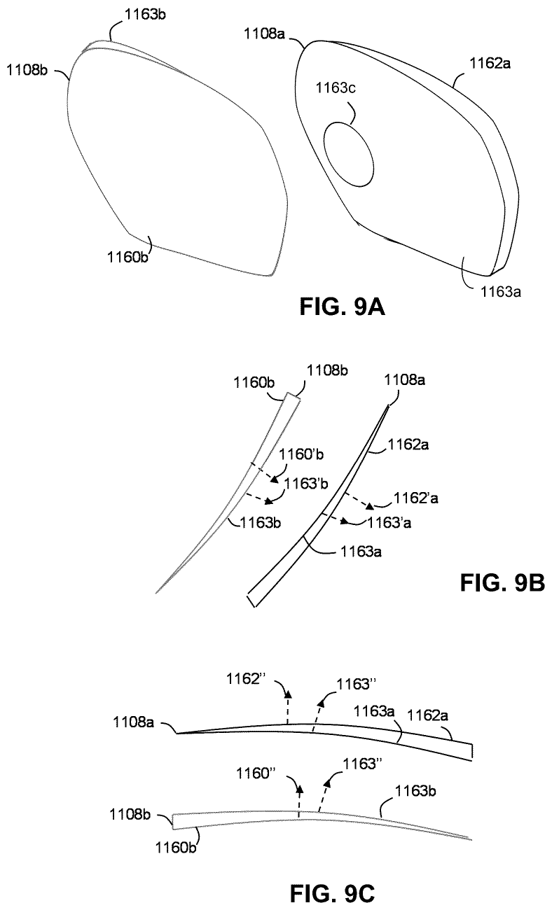

Reference is now made to FIGS. 8A, 8B, 8C, 9A, 9B, 9C. FIGS. 8A, 8B, and 8C are schematic illustrations of the partially transmissive partially reflective lens, in several viewpoints, constructed and operative in accordance with the embodiment of the disclosed technique. Particularly, FIG. 8A is a front-side view of the partially transmissive partially reflective lens 1108, showing a light reflecting zone 1163a on which the light beam along optical axis 157 impinges partially transmissive partially reflective lens 1108. FIG. 8B is a side view of the partially transmissive partially reflective lens 1108, showing the pantoscopic angle 1163′ of the reflective surface 1163 that is configured and operative to reflect the light beam along optical axis 157 toward the eye of the user. FIG. 8C shows a wrap angle of the inner surface, a wrap angle of the outer surface 1162, and a wrap angle of the intermediate reflective surface of partially transmissive partially reflective lens. With reference to FIG. 8B, a typical pantoscopic angle 1163′ is in the range of 20 to 25 degrees. Furthermore, FIG. 8B shows a pantoscopic angle 1160′ of inner surface 1160 and a pantoscopic angle 1162′ of outer surface 1162, which differs from pantoscopic angle 1163′ of the intermediate reflective surface 1163. The pantoscopic angles of both inner 1160 and outer surfaces 1162 form a mechanical structure of the partially transmissive partially reflective lens 1108, thereby facilitating in defining the pantoscopic angle of partially transmissive partially reflective lens 1108. The maximal pantoscopic angle of inner and outer surfaces 1160 and 1162 (respectively) relative to intermediate reflective surface 1163 is limited by the overall size and thickness of the partially transmissive partially reflective lens 1108. Typical pantoscopic angles 1160′ and 1162′ can be reduced by around 2 degrees (not shown) with respect to reflective surface pantoscopic angle 1163′. FIG. 8C shows a top-side view of the partially transmissive partially reflective lens 1108, showing a wrap angle 1163″ of the intermediate reflective surface 1163 that is operative and configured to reflect the light beam along optical axis 157 toward the eye of the user. Furthermore, FIG. 8C shows a wrap angle 1160″ of inner surface 1160 and the wrap angle 1162″ of outer surface 1162, which differs from wrap angle 1163″ of intermediate reflective surface 1163. The wrap angles of both inner and outer surfaces (1160 and 1162, respectively form a mechanical structure of the partially transmissive partially reflective lens 1108, thus defining the apparent wrap angle of partially transmissive partially reflective lens 1108. A typical wrap angle is in the range of between 12 and 18 degrees. The tilt and wrap angles of the inner and outer surfaces may be defined to meet particular optical designs and/or standards, irrespective of the tilt and wrap angles of the intermediate reflective surface, thus enabling to set the pantoscopic and wrap angles of the partially transmissive partially reflective lens (e.g., to meet common standards (e.g., a pantoscopic angle of 15 degrees)). The inner and outer radii of curvatures may each be defined and formed independently to allow the correction of prismatic deviation, when the user looks through the lens.

Now with reference to FIGS. 9A, 9B, and 9C, which are schematic illustrations in an exploded view detailing the structure of the partially transmissive partially reflective lens shown in FIGS. 8A, 8B, and 8C. Specifically, FIG. 9A is a perspective exploded view showing the construction of the partially transmissive partially reflective lens 1108 from two main parts. A first part 1108a is constructed from two subparts, namely, an outer surface 1162a, and a reflective surface 1163a. Reflective surface 1163a includes a reflective zone 1163c (shown to be circular) (the reflective zone has an area that is smaller than the area of intermediate reflective surface 1163). A second part 1108b is constructed from two subparts, namely, an inner surface 1160b, and reflective surface 1163b. Both first part 1108a and second part 1108b are made of the same material (e.g., polycarbonate). Intermediate reflective surfaces 1163a and 1163b, of parts 1108a and 1108b (respectively), match with each other with respect to having the same radii of curvatures and the same tilt angles. Intermediate reflective surface 1163a is coated with a partially transmissive partially reflective layer (not shown). Typical transmissive/reflection values are in the range of 90-60/10-40 respectively. First and second parts 1108 a and 1108 b are coupled to each other (e.g., glued with an optical grade adhesive). Alternatively, intermediate reflective surface 1163b is coated with a partially transmissive partially reflective layer (not shown) instead of reflective surface 1163a to attain the same level of reflectivity of reflective surface 1163a. According to a particular implementation the reflectivity layer is implemented in a designated area or zone (e.g., a circle zone, as shown in FIG. 9A), namely at reflective zone 1163c, where the light display originated light beam impinges reflective surfaces 1163a and 1163b. The light beam from the light display traverses back and forth through second part 1108b, which is constructed from an optical clear grade material to avoid or at least minimize light (illumination) losses. FIGS. 9B and 9C show the side and top exploded views of the first and second parts 1108a and 1108b. Specifically, FIGS. 9B and 9C show the mechanical structures of both first and second parts 1108a and 1108b, which are characterized by a variable thickness. According to another particular implementation, where tinted visible light transmittance (VLT) is required, a variable (e.g., gradient) tint coating is applied in order to compensate for thickness variations along the mechanical structure of partially transmissive partially reflective lens 1108.

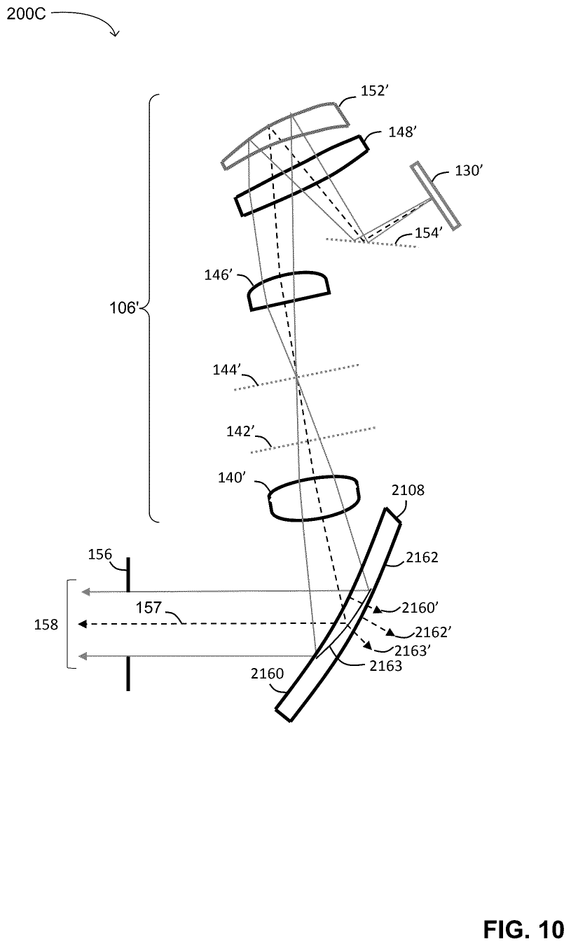

Reference is now made to FIG. 10, which is an illustration showing a schematic diagram of a light path through a glabella-region positioned optical module of an electro-optical unit in an optical see-through (OST) near-eye display (NED) off-axis system for improving a pantoscopic angle, generally referenced 200C, constructed and operative in accordance with a further embodiment of the disclosed technique. All elements of system 200C are the same as those respective elements of system 200A, except for those elements referenced, and numbered differently. Specifically, system 200C includes a partially transmissive partially reflective lens 2108 (different from 1108 of system 200A (FIG. 7A) and system 200B (FIG. 7B)), an electro-optical unit (of FIG. 7A), which in turn includes an optical module 106′.

Partially transmissive partially reflective lens 2108 includes an inner surface 2160 exhibiting an inner radius of curvature with a tilt angle 2160′, an outer surface 2162 exhibiting an outer radius of curvature with a tilt angle 2162′, and an intermediate reflective surface 2163 (located in between the inner and outer surfaces), exhibiting an intermediate reflective radius of curvature with a tilt angle 2163′. The structure, location and size of intermediate reflective surface 2163 is optimized to the light beam 158. Light display 130′ projects light beam 158 on partially transmissive partially reflective lens 2108. Light beam 158 passes through inner surface 2160, refracts, i.e., propagates through the refractive material of partially transmissive partially reflective lens 2108, reflects back from intermediate reflective surface 2163, propagates again through the material of partially transmissive partially reflective lens 2108, passes through inner surface 2160 and propagates through the air towards the eye of the user along optical axis 157. Intermediate reflective surface 2163 extends partially along the lengthwise dimension (length) of partially transmissive partially reflective lens 2108 (e.g., approximately a third of the length of lens 2108). An upper end of intermediate reflective surface 2163 is adjacent to outer surface 2162, and an lower end of intermediate reflective surface 2163 is adjacent to inner surface 2160, thereby delineating tilt angle 2163′. Alternatively, intermediate reflective surface 2163 exhibits other lengths (not shown) in comparison to the length of partially transmissive partially reflective lens 2108 (e.g., length ratios) (e.g., half, quarter, etc.).

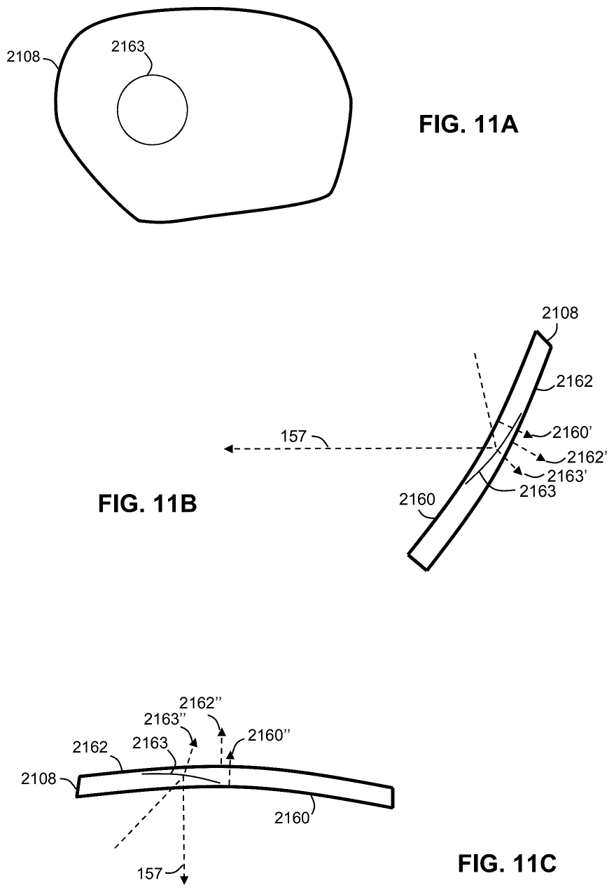

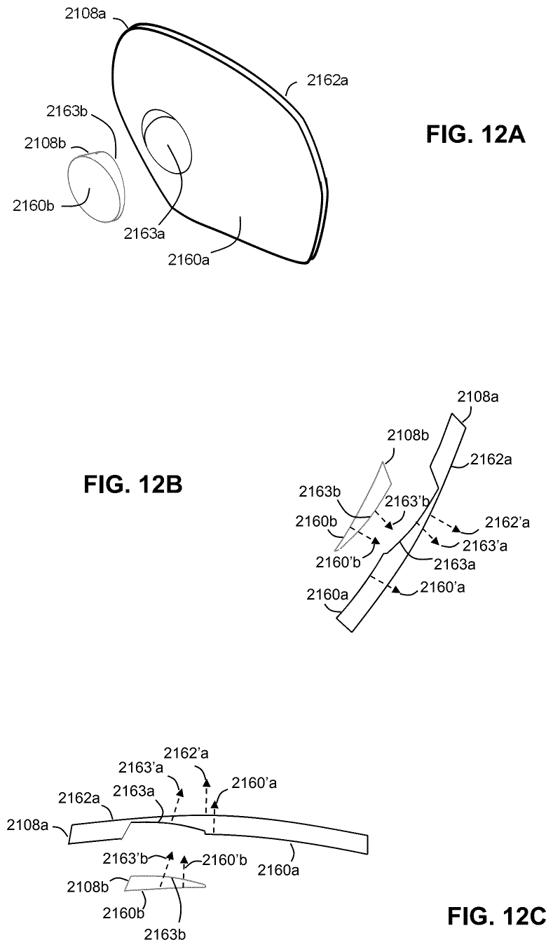

Reference is now made to FIGS. 11A, 11B, 11C, 12A, 12B, 12C. FIGS. 11A to 11C are schematic illustrations of the partially transmissive partially reflective lens, in several viewpoints, constructed and operative in accordance with the embodiment of the disclosed technique. Particularly, FIG. 11A is a front view of a partially transmissive partially reflective lens, showing a reflective zone at which a light beam along an optical axis impinges the partially transmissive partially reflective lens. FIG. 11B is a side view of the partially transmissive partially reflective lens, showing a tilt angle of an intermediate reflective surface configured and operative to reflect a light beam along the optical axis toward the eye of the user. FIG. 11C is a top view of the partially transmissive partially reflective lens, showing a wrap angle of the intermediate reflective surface that is operative and configured to reflect the light beam along the optical axis toward the eye of the user.

Specially, FIG. 11A illustrates a front view of the partially transmissive partially reflective lens 2108, showing a reflective zone 2163 that is configured and operative to reflect light beam 158 (FIG. 10) that impinges partially transmissive partially reflective lens 2108 along optical axis 157. FIG. 11B illustrates a side view of the partially transmissive partially reflective lens 2108, showing a pantoscopic angle 2163′ of intermediate reflective surface 2163 that is configured and operative to reflect the light beam along optical axis 157 toward the eye of the user. A typical value of pantoscopic angle 2163′ is in the range between 20 to 25 degrees. Additionally, FIG. 11B shows a pantoscopic angle 2160′ of inner surface 2160 and a pantoscopic angle 2162′ of outer surface 2162, which differs from pantoscopic angle 2163′ of the intermediate reflective surface 2163. The pantoscopic angles of both inner and outer surfaces (2160 and 2162, respectively), define the form of a mechanical structure of partially transmissive partially reflective lens 2108, thus facilitating in defining the pantoscopic angle of partially transmissive partially reflective lens 2108. The maximal pantoscopic angle of inner and outer surfaces 2160 and 2162 (respectively) relative to intermediate reflective surface 2163 is limited by the overall size of the reflective zone of surface 2163 and thickness of the partially transmissive partially reflective lens 2108. Typically, pantoscopic angles 2160′ and 2162′ can be maximally reduced by around 8 degrees, with respect to the reflective surface pantoscopic angle 2163′. FIG. 11C illustrates a top view of partially transmissive partially reflective lens 2108, exhibiting a wrap angle 2163″ of intermediate reflective surface 2163, which in turn is configured and operative to reflect the light beam along optical axis 157 toward the eye of the user. Additionally, FIG. 11C shows a wrap angle 2160″ of inner surface 2160 and a wrap angle 2162″ of outer surface 2162, which differs from a wrap angle 2163″ of the reflective surface 2163. The wrap angles of both inner and outer surfaces (2160 and 2162, respectively), define the form the mechanical structure of the partially transmissive partially reflective lens 2108, thereby defining a wrap angle of partially transmissive partially reflective lens 2108. The maximal wrap angle of inner and outer surfaces 2160 and 2162 (respectively) relative to intermediate reflective surface 2163 is limited by the overall size of the reflective zone of surface 2163 and thickness of the partially transmissive partially reflective lens 2108. Typically, the wrap angles 2160″ and 2162″ can be maximally reduced by around 8 degrees, with respect to intermediate reflective surface wrap angle 2163″.

FIGS. 12A to 12C are schematic illustrations detailing the structure in exploded views of the partially transmissive partially reflective lens of FIGS. 11A to 11C, constructed and operative in accordance with the embodiment of the disclosed technique. Specifically, FIG. 12A is a perspective exploded view showing the construction of partially transmissive partially reflective lens 2108 from two main (first and second) parts. A first part 2108a includes an outer surface 2162a, an inner surface 2160a, and a reflective surface 2163a. A second part 2108b includes an inner surface 2160b, and a reflective surface 2163b. Both first and second parts 2108a and 2108b are made of the same material (e.g., polycarbonate). Second part 2108b is shown to generally resemble a solid oblique cylindrical segment that is sliced by two curved surfaces such that inner surface 2108a and reflective surface 2163b are curved and form an angle with respect each other. First part 2108a includes a three-dimensional cut-out portion whose dimensions matches to accommodate the dimensions of second part 2108b. Particularly, reflective surfaces 2163a and 2163b, of first and second parts 2108a and 2108b, respectively, match each other by having the same radii of curvatures and the same of tilt angles. Reflective surface 2163a is coated with a partially transmissive partially reflective layer. Typical transmissive/reflection values are in the range of 90-60/10-40 respectively. First and second parts 2108 a and 2108 b are coupled together (e.g., glued to each other with an optical grade adhesive). Alternatively, reflective surface 2163b is coated the same as reflective surface 2163a to exhibit the same reflectivity. The size, shape and location of the reflective surface is configured, constructed, and designed to fit into a zone where the light beam impinges both first and second parts 2108a and 2018b. Second part 2108b is made of a clear optical grade material which is essentially the same material as part 2108a with the same refractive index to avoid, reduce, or minimize light losses or light reflections at the mating (and interfacing) surfaces between part 2108a and part 2108b, as the light beam from the light display passes back and forth through it. FIGS. 12B and 12C show the side and top exploded views (respectively) of first and second parts 2108a and 2108b (respectively). The mechanical structures of the first and second parts 2108a and 2108b are characterized as having variable thicknesses along their respective lengths, as in a case where tinted (partial) transmittance is required, a variable/gradient tint coating is implemented in order to compensate for thickness variations along the mechanical structure.

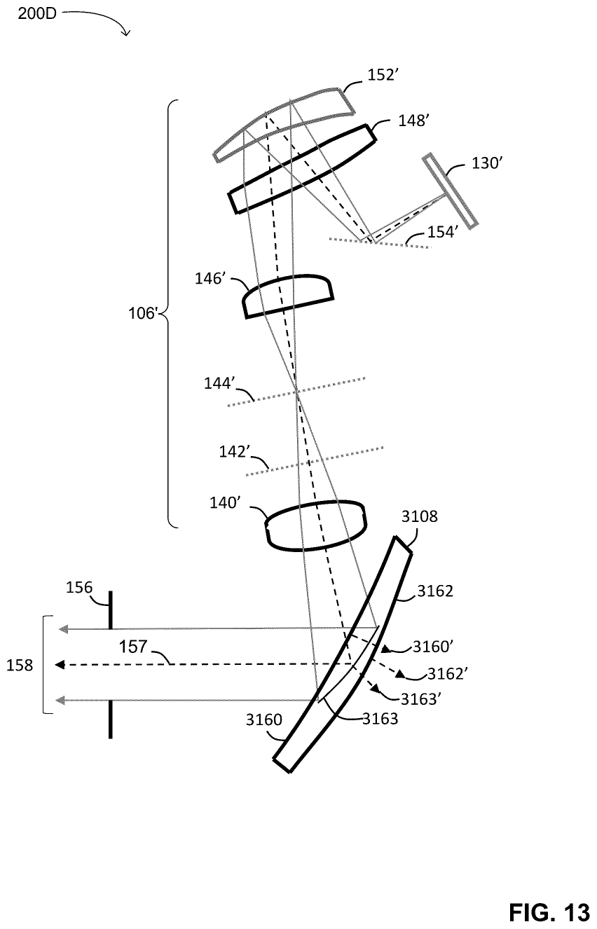

Reference is now made to FIG. 13, which is an illustration showing a schematic diagram of a light path through a glabella-region positioned optical module of an electro-optical unit in an optical see-through (OST) near-eye display (NED) off-axis system, providing ophthalmic correction the user, utilizing an intermediate reflective surface embedded in the partially transmissive partially reflective lens, generally referenced 200D, constructed and operative in accordance with another embodiment of the disclosed technique. All elements of system 200D are the same as those respective elements of system 200A, except for those elements referenced, and numbered differently. Specifically, system 200D includes a partially transmissive partially reflective lens 3108 (different from lens 1108 of system 200A (FIG. 7A) and system 200B (FIG. 7B)), an electro-optical unit (of FIG. 7A), which in turn includes an optical module 106′.

Partially transmissive partially reflective lens 3108 includes an inner surface 3160 exhibiting an inner radius of curvature and a first optical power with a tit angle of 3160′, an outer surface 3162 exhibiting an outer radius of curvature and a second optical power with a tilt angle 3162′, and an intermediate reflective surface 3163, located in between the inner and outer surfaces, exhibiting a reflective radius of curvature and a third optical power with a tilt angle 3163′. The structure, location, and size of reflective surface 3163 is optimized to light beam 158. Light display 130′ projects light beam 158 onto partially transmissive partially reflective lens 3108, by passing through inner surface 3160, refracting, i.e., propagating through the material of partially transmissive partially reflective lens 3108, reflecting back from reflecting surface 3163, refracting again through the material of partially transmissive partially reflective lens 3108, passing through inner surface 3160 and propagating through the air toward the eye of the user along optical axis 157. The focal distance of the projected light beam is varied by altering the reflective radius of curvature in conjunction with the inner radius of curvature. The focal distance at which the user sees the outside scenery through partially transmissive partially reflective lens 3108 is variable by altering the outer radius of curvature in conjunction with the inner radius of curvature. FIG. 13 schematically shows that the outer surface 3162 and inner surface 3160 fully determine the optical power for ophthalmic correction of the outward scene, while intermediate reflective surface 3163 solely determines the optical power for ophthalmic correction of the display.

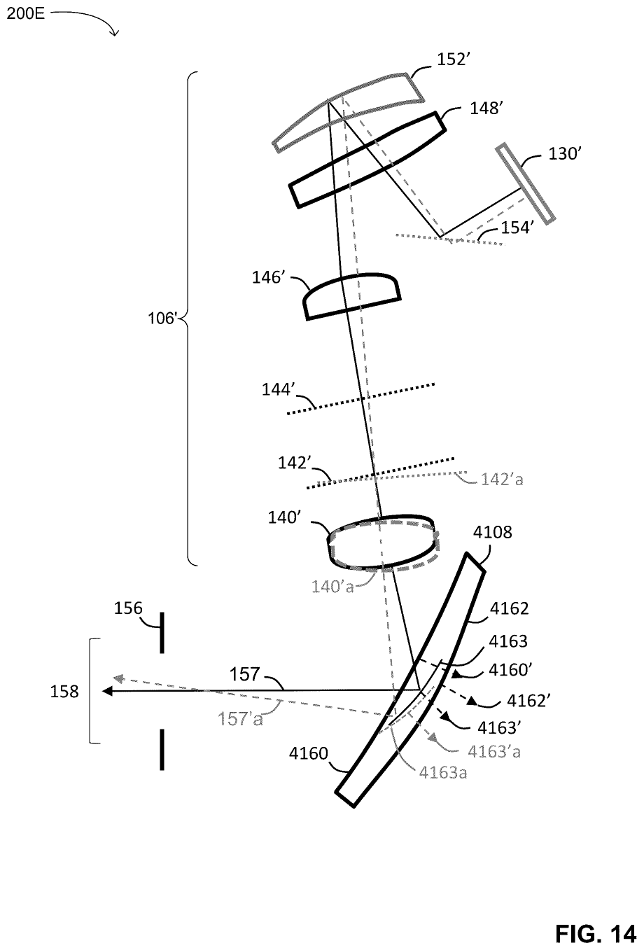

Reference is now made to FIG. 14, which is an illustration showing a schematic diagram of a light path through a glabella-region positioned optical module of an electro-optical unit in an optical see-through (OST) near-eye display (NED) off-axis system, generally referenced 200E, constructed and operative in accordance with a further embodiment of the disclosed technique. All elements of system 200E are the same as those respective elements of system 200A, except for those elements referenced, and numbered differently. Specifically, system 200E includes a partially transmissive partially reflective lens 4108 (different from lens 1108 of system 200A (FIG. 7A) and system 200B (FIG. 7B)), an electro-optical unit (of FIG. 7A), which in turn includes an optical module 106′.

Partially transmissive partially reflective lens 4108 includes an inner surface 4160 exhibiting an inner radius of curvature with a tit angle of 4160′, an outer surface 4162 exhibiting an outer radius of curvature with a tilt angle 4162′, and an intermediate reflective surface 4163, located in between the inner and outer surfaces, exhibiting a reflective radius of curvature with a tilt angle 4163′. Light display 130′ projects light beam 158 onto partially transmissive partially reflective lens 4108. Light beam 158 passes through inner surface 4160, propagates through (refracts by) the material of partially transmissive partially reflective lens 4108, reflects back from reflecting surface 4163, refracts again through the material of partially transmissive partially reflective lens 4108, passes through inner surface 4160 and propagates through the air, passing through the designed display exit pupil 156 toward the eye of the user along optical axis 157. The optical configuration shown in FIG. 14 enables the altering of the optical axis angle by changing the tilt angle 4163′ of the reflective surface 4163. For maintaining the optical axis of the light beam centered with respect to the designed display exit pupil 156, reflective surface 4163 is displaced in conjunction with first lens 140′ and reflector 142′ both being tiltable and repositionable. An optical axis 157′a shown by dotted line in FIG. 14 represents a tiltable optical axis configuration providing a tiltable direction tilted by an a angle with respect to a non-tiltable optical axis defined by optical axis 157. The optical elements in the tiltable optical axis optical configuration enabling the optical axis to be tilted at an angle α (with respect to non-tiltable optical axis configuration) are first lens 140′ (designated in the tiltable optical axis configuration 140′a), reflector 142′ (designated in the tiltable optical axis configuration 142′a), and reflective surface 4163′ (designated in the tiltable optical axis configuration 4163′a). Reflective surface 4163a is tilted by a tilt angle 4163′a and displaced with respect to the non-tiltable reflective surface 4163 tilt angle 4163′, so an optical axis 157′a is defined between the center of the reflective surface 4163a and the center of the display exit pupil along tilt angle a with respect to the non-tiltable optical axis configuration defined by optical axis 157. Reflector 142′a and first lens 140′a are tilted with respect to reflector 142 and first lens 140, (respectively) to allow light beams to propagate along optical axis 157a toward a tiltable direction aimed at the center of the reflective surface 4163a. For a change in the tilt direction of α degrees, the intermediate reflective surface is tilted from 4163′ to 4163′a by a value of α/2 degrees.

1. An optical see-through (OST) near-eye display (NED) off-axis system, providing ophthalmic correction to an eye of a user, for improving a pantoscopic angle in a system based on a glabellar region positioned electro-optical sub-system, comprising:
a partially transmissive partially reflective lens, including a material having a refractive index, an inner surface characterized by an inner surface radius of curvature exhibiting a first optical power, and an outer surface characterized by an outer surface radius of curvature exhibiting a second optical power, said partially transmissive partially reflective lens is configured to be facing said eye, and to at least partially transmit incoming light of an outward scene to said eye;
an intermediate reflective surface, characterized by an intermediate reflective radius of curvature exhibiting a third optical power, said intermediate reflective surface is embedded within said partially transmissive partially reflective lens, between said inner surface and said outer surface; and
an electro-optical sub-system, configured to be optically coupled with said partially transmissive partially reflective lens, said electro-optical sub-system including a light display configured to project a light beam image onto said intermediate reflective surface enabling reflection of said light beam image toward said eye, said electro-optical sub-system is configured to be located at a glabellar region of said user,
wherein said first optical power and said second optical power are configured, in conjunction with said refractive index, to provide ophthalmic correction with respect to transmitted said incoming light from said outward scene for viewing by said eye, wherein said third optical power is configured to provide ophthalmic correction with respect to reflected said light beam image for viewing by said eye, wherein said intermediate reflective surface is oriented such to have a tilt angle with respect to said at least one of said inner surface and said outer surface enabling a reduction of said pantoscopic angle.

2. The OST NED off-axis system according to claim 1, wherein said intermediate reflective surface is coated with at least one of a partially transmissive partially reflective mirror material, and a partially transparent partially reflective material.

3. The OST NED off-axis system according to claim 1, wherein said intermediate reflective surface is at least one of: a diffractive grating, a meta-surface; and a holographic grating.

4. The OST NED off-axis system according to claim 1, further comprising a user-attachment section that enables a detachable coupling of said OST NED off-axis system to said user.

5. The OST NED off-axis system according to claim 1, wherein said electro-optical sub-system includes an optical module that includes an off-axis Mangin mirror.

6. The OST NED off-axis system according to claim 5, wherein said off-axis Mangin mirror is configured to compensate for at least one of: optical aberrations of said partially transmissive partially reflective lens.

7. The OST NED off-axis system according to claim 5, wherein said off-axis Mangin mirror is configured to compensate for residual astigmatism aberrations caused by an off-axis optical configuration of said OST NED off-axis system.

8. The OST NED off-axis system according to claim 1, wherein at least one of said inner surface and said outer surface is coated with an anti-reflective coating to minimize residual optical aberrations that include at least one of ghosting, stray light, and residual reflections.

9. The OST NED off-axis system according to claim 1, wherein said pantoscopic angle is in a range between 20 to 25 degrees.

10. The OST NED off-axis system according to claim 1, wherein said OST NED off-axis system has a wrap angle in a range between 12 and 18 degrees.

11. The OST NED off-axis system according to claim 1, wherein said intermediate reflective surface includes a reflective zone whose area is smaller than the area of said intermediate reflective surface, said reflective zone is configured to reflect said light beam image toward said eye of said user.

12. The OST NED off-axis system according to claim 1, wherein said partially transmissive partially reflective lens is constructed from a first structural part and a second structural part, said first structural part includes a first structural part outer surface, a first structural part inner surface, and a first structural part reflective surface, said second structural part includes a second structural part inner surface and a second structural part reflective surface.

13. The OST NED off-axis system according to claim 12, wherein said first structural part reflective surface is coated with a first reflective coating material, and said second structural part reflective surface is coated with a second reflective coating material, wherein said first reflective coating material and said second reflective coating material either have identical reflectivity characteristics or different said reflectivity characteristics.

14. The OST NED off-axis system according to claim 1, wherein said intermediate reflective surface is tiltable with respect to at least one of said inner surface and said outer surface, thereby tiling an optical axis of said OST NED off-axis system.

15. The OST NED off-axis system according to claim 1, further comprising a processor configured to communicate data with said light display.

16. The OST NED off-axis system according to claim 15, further comprising a memory device configured to communicate data with at least one of said processor and said light display.

17. The OST NED off-axis system according to claim 15, further comprising a user interface configured to communicate data with said processor, and to further perform at least one of:
receive an input from said user;
transmit an output to said user;
present information to said user;
enable interaction with said user; and
communicate with said user.

18. An optical see-through (OST) near-eye display (NED) off-axis system, providing ophthalmic correction to an eye of a user, for improving a pantoscopic angle in a system based on a glabellar region positioned electro-optical sub-system, comprising:
a partially transmissive partially reflective lens, including a material having a refractive index, an inner surface, and an outer surface, characterized by said partially transmissive partially reflective lens is configured to be facing said eye, and to at least partially transmit incoming light of an outward scene to said eye;
an intermediate reflective surface, embedded within said partially transmissive partially reflective lens, between said inner surface and said outer surface; and
an electro-optical sub-system, configured to be optically coupled with said partially transmissive partially reflective lens so as to project a light beam image onto said intermediate reflective surface, said electro-optical sub-system configured to be located at a glabellar region of said user, said electro-optical sub-system including:
a light display;
a first lens;
a second lens;
a third lens; and
a fourth reflecting lens.
wherein said light beam image propagates along an optical path from said light display toward said third lens, then toward said fourth reflecting lens, then again toward said third lens, then toward said second lens, then toward said first lens, then toward said partially transmissive partially reflective lens.

19. The OST NED off-axis system according to claim 18, wherein said partially transmissive partially reflective lens refracts said light beam image toward said intermediate reflective surface that reflects said light beam image toward said partially transmissive partially reflective lens, and directs said light beam image to toward said eye.

20. The OST NED off-axis system according to claim 18, wherein said fourth reflecting lens is an off-axis Mangin mirror.

21. The OST NED off-axis system according to claim 18, wherein said inner surface is characterized by an inner surface radius of curvature exhibiting a first optical power, said outer surface is characterized by an outer surface radius of curvature exhibiting a second optical power, said intermediate reflective surface is characterized by an intermediate reflective radius of curvature exhibiting a third optical power.

22. The OST NED off-axis system according to claim 21, wherein said first optical power and said second optical power are configured in conjunction with said refractive index to provide ophthalmic correction with respect to transmitted said incoming light for viewing by said eye, wherein said third optical power is configured to provide ophthalmic correction with respect to reflected said light beam image for viewing by said eye, wherein said intermediate reflective surface is oriented such to have a tilt angle with respect to said at least one of said inner surface and said outer surface enabling a reduction of said pantoscopic angle.

23. The OST NED off-axis system according to claim 18, wherein said intermediate reflective surface is coated with at least one of a partially transmissive partially reflective mirror coating, and a partially transparent partially reflective material.

24. The OST NED off-axis system according to claim 18, wherein said intermediate reflective surface is at least one of: a diffractive grating, a meta-surface; and a holographic grating.

25. The OST NED off-axis system according to claim 20, wherein said off-axis Mangin mirror is configured to compensate for residual astigmatism aberrations caused by an off-axis optical configuration of said OST NED off-axis system.

26. The OST NED off-axis system according to claim 18, wherein at least one of said inner surface and said outer surface is coated with an anti-reflective coating to minimize residual optical aberrations that include at least one of ghosting, stray light, and residual reflections.

27. The OST NED off-axis system according to claim 18, wherein said intermediate reflective surface includes a reflective zone whose area is smaller than the area of said intermediate reflective surface, said reflective zone is configured to reflect said light beam image toward said eye of said user.

28. The OST NED off-axis system according to claim 18, wherein said partially transmissive partially reflective lens is constructed from a first structural part and a second structural part, said first structural part includes a first structural part outer surface, a first structural part inner surface, and a first structural part reflective surface, said second structural part includes a second structural part inner surface and a second structural part reflective surface.

29. The OST NED off-axis system according to claim 28, wherein said first structural part reflective surface is coated with a first reflective coating material, and said second structural part reflective surface is coated with a second reflective coating material, wherein said first reflective coating material and said second reflective coating material either have identical reflectivity characteristics or different said reflectivity characteristics.

30. The OST NED off-axis system according to claim 18, wherein said intermediate reflective surface is tiltable with respect to at least one of said inner surface and said outer surface, thereby tiling an optical axis of said OST NED off-axis system.
